SEQUENCE LISTING (10 sequences)

SEQ ID NO: 1 - protein, 5 aa, synthetic construct
  REGION 1..5: note = VHH-1 CDR1
TYTGG

SEQ ID NO: 2 - protein, 17 aa, synthetic construct
  REGION 1..17: note = VHH1 - CDR2
TFSWNSGRTHFADSVKG

SEQ ID NO: 3 - protein, 12 aa, synthetic construct
  REGION 1..12: note = VHH-1 - CDR3
HRTSLTDYSFRY

SEQ ID NO: 4 - protein, 4 aa, synthetic construct
  REGION 1..4: note = VHH-2 - CDR1
NSMA

SEQ ID NO: 5 - protein, 17 aa, synthetic construct
  REGION 1..17: note = VHH2- CDR2
AISWNSGRTDYADSVKG

SEQ ID NO: 6 - protein, 12 aa, synthetic construct
  REGION 1..12: note = VHH2- CDR3
HRVRIEPKEYLY

SEQ ID NO: 7 - protein, 121 aa, synthetic construct
  REGION 1..121: note = VHH-1
EVQLVESGGGLVQAGGSLRLSCAASGQTDSTYTGGWFRQAPGKEREFVATFSWNSGRTHFADSVKGRFTISRDNAKSTVFLQMNSLKPEDTAVYYCAAHRTSLTDYSFRYWGQGTHVTVSS

SEQ ID NO: 8 - protein, 120 aa, synthetic construct
  REGION 1..120: note = VHH-2
EVQLVESGGGLVQAGGSLRLSCAVSGRTVSNSMAWFRQAPEKEREFVAAISWNSGRTDYADSVKGRFTISRDNAKNTVDLQMNSLKAEDTAVYYCAVHRVRIEPKEYLYWGQGTQVTVSS

SEQ ID NO: 9 - protein, 128 aa, synthetic construct
  REGION 1..128: note = Neg Control VHH
QVQLQESGGGLVQAGGSLRLSCAASGRATSGHGHYGMGWFRQVPGKEREFVAAIRWSGKETWYKDSVKGRFTISRDNAKTTVYLQMNSLKPEDTAVYYCAARPVRVDDISLPVGFDYWGQGTQVTVSS

SEQ ID NO: 10 - DNA, 24 bp, synthetic construct
  misc_feature 1..24: note = primer
  source 1..24: mol_type = other DNA
gagcggataacaatttcacacagg

(19) United States
(12) Patent Application Publication
(10) Pub. No.: US 2025/0289904 A1
(43) Pub. Date: Sep. 18, 2025

(54) SINGLE DOMAIN ANTIBODIES FOR THE DETECTION OF PLASMIN-CLEAVED VWF

(71) Applicants: UMC UTRECHT HOLDING B.V., Utrecht (NL); TARGED BIOPHARMACEUTICALS B.V., Utrecht (NL)

(72) Inventors: Coen MAAS, Vught (NL); Steven DE MAAT, Utrecht (NL)

(21) Appl. No.: 18/861,425

(22) PCT Filed: May 1, 2023
(86) PCT No.: PCT/EP2023/061431

§ 371 Date: Oct. 29, 2024

(30) Foreign Application Priority Data
May 2, 2022 (EP) 22171173.2

(51) Int. Cl. C07K 16/36; G01N 33/86

(52) U.S. Cl. CPC C07K 16/36; G01N 33/86; C07K 2317/22; C07K 2317/569; G01N 2333/755; G01N 2500/02; G01N 2800/22; G01N 2800/32

(57) ABSTRACT

The present invention relates to single antibody domains, especially single antibody domains that specifically bind plasmin-cleaved von Willebrand factor (VWF). The invention further relates a method for detecting plasmin-cleaved VWF. This method further allows for the determining the activity of a therapeutic agent regulating plasmin-mediated cleavage of VWF, or the determination of a vascular event in a subject. The invention further describes kits comprising these single antibody domains.

### FIELD OF THE INVENTION

The present invention relates to the field of medicine and molecular diagnostics. In particular it relates to single antibody domains, especially single antibody domains that specifically bind plasmin-cleaved von Willebrand factor (VWF). The invention further relates a method for detecting plasmin-cleaved VWF, or for determining the activity of a therapeutic agent regulating plasmin-mediated cleavage of VWF, or for determining the occurrence of a vascular event in a subject.

### BACKGROUND OF THE INVENTION

VWF is a glycoprotein circulating in plasma as a series of multimers ranging in size from about 500 to 20,000 kD. Multimeric forms of VWF are composed of subunits of 250 kD each linked together by disulfide bonds. VWF mediates the initial platelet adhesion to the subendothelium of a damaged vessel wall, though only the largest multimers appear to exhibit haemostatic activity. Such VWF multimers having large molecular masses are stored in the Weibel Palade bodies of endothelial cells, and it is believed that mainly endothelial cells secrete these large polymeric forms of VWF. Those forms of VWF which have a low molecular weight (low molecular weight or LMW VWF) are believed to arise from proteolytic cleavage of the larger multimers.

Several proteases have been shown to be able to cleave VWF, thereby impairing its binding affinity for platelets among which is ADAMTS13 (a disintegrin and metalloproteinase with a thrombospondin type I motif, member 13). ADAMTS13 normally regulates the thrombogenicity of VWF by enzymatically reducing its multimer size. Hereto, VWF needs to unfold from its globular form into an unrolled conformation, thereby exposing its A2 domain for proteolysis. ADAMTS13 then cleaves a single peptide bond (Tyr1605-Met1606) within the A2 domain of VWF. Inherited or acquired ADAMTS13 deficiency results in the accumulation of ultra large VWF (UL-VWF) multimers in plasma. These UL-VWF can trigger the formation of pathological platelet aggregates, which obstruct the microvasculature, thereby causing thrombotic thrombocytopenia purpura (TTP).

Besides ADAMTS13, VWF can be cleaved by the enzyme plasmin (Berkowitz et al., J Clin Invest 1987 February; 79 (2): 524-31). We previously identified that systemic plasminogen activation (with streptokinase) was therapeutic in a mouse model for thrombocytopeniaurpura (TTP), suggesting that plasmin can act as a functional alternative to ADAMTS13 (Tersteeg et al., 2014, Circulation, 129 (12): 1320-31). Although plasmin (ogen) can directly bind to unrolled VWF, natural plasminogen activators-such as tissue plasminogen activator (tPA) and urokinase plasminogen activator (uPA)—cannot. The natural targets of tPA and uPA are fibrin and the endothelial cell receptor uPAR, respectively.

While much research has been conducted toward ADAMTS13 cleaved VWF and its role in disease, much less is known about the consequence of plasmin-cleaved VWF. However, it has been demonstrated that abnormal VWF fibers formed under pathological flow conditions are resistant to ADAMTS13 proteolysis, but remain susceptible to cleavage by plasmin (Herbig B A et al, 2015, J Thromb Haemost. 13:1699-170). Additionally, in vitro and in vivo studies have confirmed that plasmin can also successfully degrade UL-VWF platelet aggregates (Tersteeg supra, Wohner et al, 2012, Thromb Res 129: e41-e46). These emerging data thus show that plasmin-induced cleavage of VWF is of particular importance in specific pathological states. However, to this date, to the best of our knowledge no components and method have been describe which allow to discriminate between ADAMTS13 cleaved VWF, plasmin-cleaved VWF and intact VWF, which hinders the study of the exact role of plasmin-cleaved VWF pathological states. It is thus an object of the invention to provide for methods and binding molecules that are able to distinguish between different proteolytic cleavage products of VWF, and that preferably specifically recognize plasmin-cleaved VWF and not intact VWF.

### SUMMARY OF THE INVENTION

In a first aspect, the invention relates to a single variable domain comprising a combination of complementary determining region (CDR) sequences. The CDR sequences comprises a CDR3 sequence having at least 95% sequence identity with the amino acid sequence of SEQ ID NO: 6 or the amino acid sequence of SEQ ID NO:3.

In one embodiment, the CDR sequences further comprises a CDR1 sequence having at least 95% sequence identity with the amino acid sequence of SEQ ID NO: 4 or the amino acid sequence of SEQ ID NO:1. In another embodiment, wherein the CDR sequences further comprises a CDR2 sequence having at least 95% sequence identity with the amino acid sequence of SEQ ID NO: 5 or the amino acid sequence of SEQ ID NO:2. In one preferred embodiment, the CDR sequences comprises a CDR3 sequence having at least 95% sequence identity with the amino acid sequence of SEQ ID NO: 6, together with a CDR1 sequence having at least 95% sequence identity with the amino acid sequence of SEQ ID NO: 4 and/or a CDR2 sequence having at least 95% sequence identity with the amino acid sequence of SEQ ID NO: 5. In another preferred embodiment, the tCDR sequences comprises a CDR3 sequence having at least 95% sequence identity with the amino acid sequence of SEQ ID NO: 3, together with a CDR1 sequence having at least 95% sequence identity with the amino acid sequence of SEQ ID NO: 1 and/or a CDR2 sequence having at least 95% sequence identity with the amino acid sequence of SEQ ID NO: 2.

In one embodiment, the single variable domain comprises or consists of an amino acid sequence that has at least 95% sequence identity with SEQ ID NO: 8, or an amino acid sequence that has at least 95% sequence identity with SEQ ID NO: 7.

Preferably, the single viable domain specifically binds to plasmin-cleaved VWF, more preferably human VWF. For example, the affinity of the single variable domain for plasmin-cleaved VWF is at least 100-fold higher than that for uncleaved VWF. Alternatively or additionally, the affinity of the single variable domain for plasmin-cleaved VWF is at least 10-fold higher than that for ADAMTS13 cleaved VWF.

In a second aspect, the invention provides a single domain antibody comprising or consisting of the single variable domain as described herein. The single domain antibody is derived from a non-human source, preferably a camelid, more preferably a camel or llama monoclonal single domain antibody.

In one embodiment, the single domain antibody or the single variable domain of the present invention is codon optimized, for example humanised or deimmunized.

In a third aspect, the invention provides a polypeptide comprising the single variable domain as described herein.

In a fourth aspect, the invention provides the single variable domain as described herein, or the single domain antibody as described herein, or the polypeptide as described herein, for use as a biomarker for plasmin-cleaved VWF, or for use in a bioassay detecting, quantitatively or qualitatively, plasmin-cleaved VWF. Preferably, the VWF is a human VWF.

In a fifth aspect, the invention provides an in vitro method for detecting plasmin-cleaved VWF in a subject, or for determining the activity of a therapeutic agent regulating plasmin-mediated cleavage of VWF in a subject. The method comprises: i) providing a sample from the subject; ii) contacting the sample with the single variable domain as described herein, or the single domain antibody as described herein, or the polypeptide as described herein; and iii) analysing the product of step ii) to determine the presence and/or the amount of plasmin-cleaved VWF contained in the sample.

In a sixth aspect, the invention provides a method for determining the occurrence of a vascular event in a subject. The method comprises: i) assaying a sample from said subject with the single variable domain as described herein, or the single domain antibody as described herein, or the polypeptide as described herein, to determine a concentration of plasmin-cleaved VWF contained in the sample; and ii) determining the occurrence of a vascular event in the subject if the concentration of plasmin-cleaved VWF is at least 4 ng/mL.

In a seventh aspect, the invention provides a nucleic acid encoding the polypeptide as described herein.

In an eighth aspect, the invention relates to a kit comprising an element. The element comprises the single variable domain as described herein, or the single domain antibody as described herein, or the polypeptide as described herein. The kit further comprises means for detecting the binding of the element to plasmin-cleaved VWF. Optionally, the kit further comprises means for collecting a sample from a subject.

Optionally, the sample is a blood sample, a serum sample, a blood plasma sample, or a urine sample. Optionally the subject is a human, a mice, a rat, or a guinea pig.

It will of course be appreciated that features described in relation to one aspect of the present invention may be incorporated into other aspects of the present invention. For example, the method of the invention may incorporate any of the features described with reference to the products of the invention and vice versa.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” and “similarity” also means the degree of sequence relatedness between amino acid or nucleic acid sequences, as the case may be, as determined by the match between strings of such sequences. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The terms “protein” or “polypeptide” are used interchangeably and refer to molecules consisting of a chain of amino acids, without reference to a specific mode of action, size, 3-dimensional structure or origin.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein. The term heterologous also applies to non-natural combinations of nucleic acid or amino acid sequences, i.e. combinations where at least two of the combined sequences are foreign with respect to each other.

Unless indicated otherwise, the terms “immunoglobulin” and “antibody” whether it used herein to refer to a single domain antibody or to a conventional 4-chain antibody is used as a general term to include both the full-size antibody, the individual chains thereof, as well as all parts, domains or fragments thereof (including but not limited to antigen-binding domains or fragments). In addition, the term “sequence” as used herein (for example in terms like “immunoglobulin sequence”, “antibody sequence”, “variable domain sequence” or “protein sequence”), should generally be understood to include both the relevant amino acid sequence as well as nucleic acid sequences or nucleotide sequences encoding the same, unless the context requires a more specific interpretation.

A “single domain antibody” is an antibody or antibody fragment consisting only of heavy chains and devoid of light chains as are known e.g. from Camelids. A single domain antibody is thus an antibody comprising a “single variable domain” wherein the antigen binding site is present on, and formed by, the single variable domain (also referred to as an “immunoglobulin single variable domain” or “ISVD”). This sets single variable domains apart from “conventional” immunoglobulins or their fragments, wherein two immunoglobulin domains, typically a heavy chain variable domain (VH) and a light chain variable domain (VL), interact to form an antigen binding site. The term “single domain antibody” as used herein includes antibodies or antibody fragments comprising the single variable domains of camelid heavy chain antibodies (VHHs), also referred to as nanobodies, domain antibodies (dAbs), and single domain antibodies derived from shark (IgNAR domains). The term “single domain antibody” as used herein can refer to polypeptides either comprising or consisting of a single variable domain.

Generally, single variable domains will be amino acid sequences that essentially consist of 4 framework regions (FR1 to FR4 respectively) and 3 complementarity determining regions (CDR1 to CDR3 respectively). Such single variable domains and fragments are most preferably such that they comprise an immunoglobulin fold or are capable for forming, under suitable conditions, an immunoglobulin fold. Thus, the amino acid sequence and structure of an immunoglobulin variable domain sequence, in particular an single variable domain can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. The CDRs may also be referred to as “hypervariable regions” (HVRs). The total number of amino acid residues in an single variable domain can be in the region of 110-120, is preferably 112-115, and is most preferably 113. It should however be noted that parts, fragments, analogs or derivatives of an single variable domain are not particularly limited as to their length and/or size, as long as such parts, fragments, analogs or derivatives meet the further requirements outlined herein and are also preferably suitable for the purposes described herein.

Thus, in the meaning of the present invention, the term “single domain antibody” comprises polypeptides which are derived from a non-human source, preferably a camelid, preferably a camel heavy chain antibody. They may be humanized, as previously described, e.g. in WO 08/101985 and WO 08/142164. Moreover, the term comprises polypeptides derived from non-camelid sources, e.g. mouse or human, which have been “camelized”, as previously described, e.g. in WO 08/101985 and WO 08/142164. The term “single domain antibody” encompasses immunoglobulin sequences of different origin, comprising mouse, rat, rabbit, donkey, human and camelid immunoglobulin sequences. It also includes fully human, humanized or chimeric immunoglobulin sequences. For example, it comprises camelid immunoglobulin sequences and humanized camelid immunoglobulin sequences, or camelized single variable domains, e.g. camelized dAb as described by Ward et al (see for example WO 94/04678 and Davies and Riechmann 1994, Febs Lett. 339:285 and 1996, Protein Engineering 9:531).

The amino acid residues of a single variable domain (or conventional variable domain) are numbered according to the general numbering for V_{H} domains given by Kabat et al. (“Sequence of proteins of immunological interest”, US Public Health Services, NIH Bethesda, Md., Publication No. 91), as applied to VHH domains from Camelids by Riechmann and Muyldermans (1999, J. Immunol. Methods; 231:25-38; see for example FIG. 2 of said reference). According to this numbering, FR1 of a VHH comprises the amino acid residues at positions 1-30, CDR1 of a VHH comprises the amino acid residues at positions 31-36, FR2 of a VHH comprises the amino acids at positions 36-49, CDR2 of a VHH comprises the amino acid residues at positions 50-65, FR3 of a VHH comprises the amino acid residues at positions 66-94, CDR3 of a VHH comprises the amino acid residues at positions 95-102, and FR4 of a VHH comprises the amino acid residues at positions 103-113. In this respect, it should be noted that as is well known in the art for V_{H} domains and for VHH domains the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDRs, position 1 according to the Kabat numbering corresponds to the start of FR1 and visa versa, position 36 according to the Kabat numbering corresponds to the start of FR2 and visa versa, position 66 according to the Kabat numbering corresponds to the start of FR3 and visa versa, and position 103 according to the Kabat numbering corresponds to the start of FR4.

Alternative methods for numbering the amino acid residues of V_{H} domains, which methods can also be applied in an analogous manner to VHH domains from Camelids, are the method described by Chothia et al. (1989, Nature 342, 877-883), the so-called “AbM definition” and the so-called “contact definition”. However, in the present description, claims and figures, the numbering according to Kabat as applied to VHH domains by Riechmann and Muyldermans will be followed, unless indicated otherwise.

For a general description of single domain antibodies and the single variable domains (VHH domains) thereof, reference is inter alia made to the following references, which are mentioned as general background art: WO 94/04678, WO 95/04079, WO 96/34103, WO 94/25591, WO 99/37681, WO 00/40968, WO 00/43507, WO 00/65057, WO 01/40310, WO 01/44301, EP 1134231, WO 02/48193, WO 97/49805, WO 01/21817, WO 03/035694, WO 03/054016, WO 03/055527 WO 03/050531, WO 01/90190, WO 03/025020; WO 04/041867, WO 04/041862, WO 04/041865, WO 04/041863 and WO 04/062551 and Hassanzadeh-Ghassabeh et al. (2013, Nanomedicine, 8 (6): 1013-1026).

Generally, it should be noted that the term “single variable domain” or “single domain antibody” as used herein in its broadest sense is not limited to a specific biological source or to a specific method of preparation. For example, single variable domain as used in the invention can be obtained (1) by isolating the single variable domain of a naturally occurring single domain antibody; (2) by expression of a nucleotide sequence encoding a naturally occurring single variable domain; (3) by “humanization” (as described below) of a naturally occurring single variable domain or by expression of a nucleic acid encoding a such humanized single variable domain; (4) by “camelization” of a naturally occurring V_{H} domain from any animal species, in particular a species of mammal, such as from a human being, or by expression of a nucleic acid encoding such a camelized V_{H} domain; (5) using synthetic or semi-synthetic techniques for preparing proteins, polypeptides or other amino acid sequences; (6) by preparing a nucleic acid encoding a single variable domain using techniques for nucleic acid synthesis, followed by expression of the nucleic acid thus obtained; and/or (7) by any combination of the foregoing. Suitable methods and techniques for performing the foregoing are state of the art and therefore known to the skilled person.

The term “specificity” refers to the number of different types of antigens or antigenic determinants to which a particular immunoglobulin sequence, antigen-binding molecule or antigen-binding protein (such as a single domain antibody of the invention) can bind. The specificity of an antigen-binding molecule can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein. Alternatively, the affinity can also be expressed as the affinity constant (KA), which is 1/K_{D}. Affinity can be determined in a manner known per se, depending on the specific combination of antigen-binding protein and antigen of interest. Avidity is herein understood to refer to the strength of binding of a target molecule with multiple binding sites by a larger complex of binding agents, i.e. the strength of binding of multivalent binding. Avidity is related to both the affinity between an antigenic determinant and its antigen-binding site on the antigen-binding molecule and the number of binding sites present on the antigen-binding molecule. Affinity, on the other hand refers to simple monovalent receptor ligand systems. A variety of methods of measuring binding affinity are known in the art, any of which can be used for purposes of the present invention. Specific illustrative embodiments are described in the following.

An “on-rate” or “rate of association” or “association rate” or “Kon” according to this invention can also be determined with the same surface plasmon resonance technique described above using a BIAcore™-2000 or a BIAcore™-3000 (BIAcore, Inc., Piscataway, NJ) as described above.

Any reference to nucleotide or amino acid sequences accessible in public sequence databases herein refers to the version of the sequence entry as available on the filing date of this document.

### DETAILED DESCRIPTION OF THE INVENTION

The present inventors have surprisingly found binding molecules that are not only able to discriminate between plasmin-cleaved VWF and intact VWF, but also between plasmin-cleaved VWF and ADAMTS13-cleaved VWF.

The term “binding molecule” as used herein indicates any molecule capable of specifically binding to plasmin-cleaved VWF. A “binding molecule,” can thus e.g. be an antibody (monoclonal or polyclonal) or antigen-binding fragment thereof, aptamer, affimer (peptide aptamer), receptor binding domain, or designed ankyrin repeat proteins (DARPins). The term “antigen-binding fragments” means a portion of the binding molecule that is capable of specifically binding the antigen, i.e. target molecule. Examples of such binding molecules include antibodies, such as a human antibody, a humanized antibody; a chimeric antibody; a recombinant antibody; a single chain antibody; a diabody; a triabody; a tetrabody; a Fab fragment; a F(ab′) 2 fragment; an IgD antibody; an IgE antibody; an IgM antibody; an IgG1 antibody; an IgG2 antibody; an IgG3 antibody; or an IgG4 antibody, and fragments thereof. In certain embodiments, the binding molecule is an antibody or an antigen-binding fragment thereof. Preferably, the antibody comprises or consists of a single variable domain.

In certain embodiments, the binding molecule of the present invention is a single variable domain which comprises a combination of complementary determining region (CDR). The CDR sequences comprises a CDR3 sequence having at least 95% sequence identity with the amino acid sequence of SEQ ID NO: 6 (HRVRIEPKEYLY) or the amino acid sequence of SEQ ID NO:3 (HRTSLTDYSFRY). Optionally, the CDR3 sequence comprises or consists of the amino acid sequence of SEQ ID NO: 6 or the amino acid sequence of SEQ ID NO:3. Preferably, the CDR3 sequence has at least 95% sequence identity with the amino acid sequence of SEQ ID NO: 6. More preferably, the CDR3 sequence comprises or consists of the amino acid sequence of SEQ ID NO: 6. Optionally, the CDR sequences further comprises a CDR1 sequence having at least 95% sequence identity with the amino acid sequence of SEQ ID NO: 4 (NSMA) or the amino acid sequence of SEQ ID NO:1 (TYTGG). Optionally, the CDR1 sequence comprises or consists of the amino acid sequence of SEQ ID NO: 4 (NSMA) or the amino acid sequence of SEQ ID NO:1 (TYTGG). The CDR sequences may further comprises a CDR2 sequence having at least 95% sequence identity with the amino acid sequence of SEQ ID NO: 5 (AISWNSGRTDYADSVKG) or the amino acid sequence of SEQ ID NO:2 (TFSWNSGRTHFADSVKG). Optionally, the CDR2 sequence comprises or consists of the amino acid sequence of SEQ ID NO: 5 (AISWNSGRTDYADSVKG) or the amino acid sequence of SEQ ID NO: 2 (TFSWNSGRTHFADSVKG). In one embodiment, the single variable domain comprises a combination of complementary determining region (CDR) sequences comprising or consisting of 1) a CDR1 comprising or consisting of: TYTGG (SEQ ID NO: 1); 2) a CDR2 comprising of consisting of: TFSWNSGRTHFADSVKG (SEQ ID NO: 2), and, 3) a CDR3 comprising of consisting of: HRTSLTDYSFRY (SEQ ID NO: 3). A single variable domain of a preferred embodiment of the invention comprises a combination of complementary determining region (CDR) sequences comprising or consisting of 1) a CDR1 comprising or consisting of: TYTGG (SEQ ID NO: 4); 2) a CDR2 comprising of consisting of: TFSWNSGRTHFADSVKG (SEQ ID NO: 5), and, 3) a CDR3 comprising of consisting of: HRTSLTDYSFRY (SEQ ID NO: 6).

In certain embodiments, the single variable domain comprises or consist of an amino acid sequence that has at least 70, 75, 80, 85, 90, 91, 92, 93, 94, 95, 96, 97, 98 or 99% sequence identity with the amino acid sequence of SEQ ID NO: 7. A single variable domain of a preferred embodiment of the invention comprises or consist of an amino acid sequence that has at least 70, 75, 80, 85, 90, 91, 92, 93, 94, 95, 96, 97, 98 or 99% sequence identity with the amino acid sequence of SEQ ID NO: 8.

Preferably, the binding molecule, for example the single variable domain, of the present invention specifically binds to plasmin-cleaved VWF. The term “VWF” or “recombinant VWF” or “rVWF” may be used interchangeably herein and refers to von Willebrand factor polypeptide. The term “cVWF” as used herein refers to plasmin-cleaved VWF.

As used herein, the term “specific binding” or “specifically binds to” or “binds to” or is “specific for” plasmin-cleaved VWF, means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labelled target. In this case, specific binding is indicated if the binding of the labelled target to a probe is competitively inhibited by excess unlabeled target. The term “specific binding” or “specifically binds to” or is “specific for” a plasmin-cleaved VWF can be exhibited, for example, by a molecule having a Kd for the target (which may be determined as described above) of at least about 10-4 M, alternatively at least about 10.5 M, alternatively at least about 10-6 M, alternatively at least about 10-7 M, alternatively at least about 10-8 M, alternatively at least about 10-9 M, alternatively at least about 10-10 M, alternatively at least about 10-11 M, alternatively at least about 10-12 M, or greater. In one embodiment, the term “specific binding” refers to binding where a binding molecule specifically binds to a plasmin-cleaved VWF without substantially binding to intact VWF. Any Kd value greater than 10-4 M (i.e. less than 100 M) is generally considered to indicate non-specific binding.

A “Kd” or “Kd value” can be measured by using an ELISA as described in the Examples herein or by using surface plasmon resonance assays using a BIAcore™-2000 or a BIAcore™-3000 (BIAcore, Inc., Piscataway, NJ) at 25° C. with immobilized antigen CM5 chips at ˜10-50 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 μg/ml (˜0.2 μM) before injection at a flow rate of 5 μl/minute to achieve approximately 10 response units (RU) of coupled protein. Following the injection of antigen, 1M ethanolamine is injected to block unreacted groups. For kinetics measurements, two-fold serial dilutions of the antibody or Fab (0.78 nM to 500 nM) are injected in PBS with 0.05% Tween 20 (PBST) at 25° C. at a flow rate of approximately 25 μl/min. Association rates (kon) and dissociation rates (Koff) are calculated using a simple one-to-one Langmuir binding model (BIAcore Evaluation Software version 3.2) by simultaneous fitting the association and dissociation sensorgram. The equilibrium dissociation constant (K_{D}) is calculated as the ratio Koff/kon. See, e.g., Chen, Y., et al., (1999) J. Mol Biol 293:865-881. If the on-rate exceeds 106 M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stir red cuvette.

In certain embodiments, the affinity of the binding molecule, for example the single variable domain, of the present invention for plasmin-cleaved VWF is at least 50-fold, 100-fold, 150-fold, 200-fold, 300-fold, 400-fold, 500-fold, 600-fold, 700-fold, 800-fold, 900-fold, 1000-fold, 1100-fold, 1200-fold higher than uncleaved VWF (intact VWF). In one embodiment, the binding molecule binds with a 144-fold higher affinity to plasmin-cleaved VWF than to intact VWF. In another embodiment, the binding molecule binds with a 1251-fold higher affinity to plasmin-cleaved VWF than to intact VWF. Alternatively or additionally, the affinity of the binding molecule, for example the single variable domain, of the present invention for plasmin-cleaved VWF is at least 2-fold, 3-fold, 4-fold, 5-fold, 6-fold, 7-fold, 8-fold, 9-fold, 10-fold, 12-fold, 15-fold, 20-fold, 25-fold, 30-fold, 40-fold, 50-fold, 60-fold, 70-fold, 80-fold, 90-fold, 100-fold higher than ADAMTS13-cleaved VWF.

In certain embodiments, the binding molecule of the present invention is a polypeptide comprising at least one single variable domain as described herein. In one embodiment, the polypeptide comprises, in addition to the at least one single variable domain of the invention, one or more amino acids.

In one embodiment, the polypeptide comprises more than one copies of the single variable domain of the invention, e.g. a bivalent or trivalent polypeptide. In such a multivalent polypeptide the single variable domains can be fused directly to each other or via a suitable linker. Suitable linker-amino acid sequences are known in the art (e.g. from Chen et al., 2013, Adv Drug Deliv Rev. 65 (10): 1357-1369). Suitable linkers are usually flexible amino acid sequences that are applied when the joined domains require a certain degree of movement or interaction. They are generally composed of small, non-polar (e.g. Gly) or polar (e.g. Ser or Thr) amino acids. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. Preferred flexible linkers have sequences consisting primarily of stretches of Gly and Ser residues (“GS” linker). An example of preferred (and widely used) flexible linker has the sequence of (Gly-Gly-Gly-Gly-Ser) n. By adjusting the copy number “n”, the length of this GS linker can be optimized to achieve appropriate separation of the functional domains, or to maintain necessary inter-domain interactions.

In one embodiment, the polypeptide comprises at least one single variable domain of the invention, fused onto at least part of a heavy chain constant region of an antibody. Preferably, the (at least part of a) heavy chain constant region is of a conventional antibody, more preferably of a human conventional antibody. Thus, in one embodiment, the (at least part of a) heavy chain constant region is of an IgG1, IgG2, IgG3 or IgG4 antibody, preferably a human IgG1, IgG2, IgG3 or IgG4 antibody. In one embodiment, the (at least part of a) heavy chain constant region comprises or consists of the CH2 and CH3 domains, wherein preferably the CH2 and CH3 domains are preceded by a hinge region.

In one embodiment, the polypeptide comprises a tag. The tag sequence may be useful for immobilizing the polypeptide onto a solid phase. Thus, the present invention also encompasses polypeptide which are immobilized onto a solid phase using such tag sequences. The tag sequence can include, but are not limited to, proteins (for example, glutathione transferase, luciferase, beta-galactosidase), peptides (for example, His tags), coupling agents (for example, carbodiimide reagents), and various kinds of labels (for example, radioactive labels, chromophores, and enzymes).

In one embodiment, the polypeptide is a single domain antibody. The single domain antibody may be derived from a non-human source, preferably a camelid. In a preferred embodiment, the single domain antibody is a llama monoclonal single domain antibody. In an alternative embodiment, the single domain antibody is a camel monoclonal single domain antibody.

The binding molecule of the present invention may be used as a biomarker for plasmin-cleaved VWF, for example, in any of the methods described herein. The binding molecule of the present invention may also be used in a bioassay detecting, quantitatively or qualitatively, plasmin-cleaved VWF.

The term “visualize” or “detect” are used interchangeably herein when discussing the examination of VWF cleavage fragment level(s) in an immunoassay, such as an ELISA, EMIT, RIA, protein microarray, immunoblot or Western blot. Likewise, the term “level” or “levels” refers to the amount or concentration of VWF visualized, detected, or measured in a blot or assay.

The present invention also provides a nucleic acid molecule comprising a nucleotide sequence encoding a binding molecule, for example the single variable domain, or the polypeptide or the antibody of the invention. The nucleotide sequence optionally encodes a signal peptide operably linked to the fusion protein. Preferably, the nucleic acid further comprises regulatory elements for (or conducive to) the expression of the polypeptide or antibody in an appropriate host cell, which regulatory elements are operably linked to the nucleotide sequence.

The present invention also provides a host cell comprising the nucleic acid molecule comprising the nucleotide sequence described herein. In one embodiment, the host cell is an isolated cell or a cultured cell. Among the host cells that may be employed are prokaryotes, yeast or higher eukaryotic cells. Prokaryotes include gram negative or gram positive organisms, for example Escherichia coli or bacilli. Suitable yeast cells include Saccharomyces cerevisiae and Pichia pastoris. Higher eukaryotic cells include insect cells and established cell lines of mammalian origin. Examples of suitable mammalian host cell lines include the COS-7 line of monkey kidney cells (Gluzman et al., 1981, Cell 23:175), L cells, HEK 293 cells, C127 cells, 3T3 cells, Chinese hamster ovary (CHO) cells, Hela cells, BHK cell lines, and the CVI/EBNA cell line derived from the African green monkey kidney cell line CVI as described by McMahan et al., 1991, EMBO J. 10:2821. Appropriate cloning and expression vectors for use with bacterial, fungal, yeast, and mammalian cellular hosts are described by Pouwels et al. (Cloning Vectors: A Laboratory Manual, Elsevier, New York, 1985). Host cells comprising the nucleic acid molecule of the invention can be cultured under conditions that promote expression of the polypeptide or antibody. Thus a further aspect the invention relates to a method for producing a binding molecule of the invention, the method comprising the step of cultivating a host cell a nucleic acid molecule comprising the nucleotide sequence encoding the binding molecule, under conditions conducive to expression of the binding molecule, and optionally, recovering the binding molecule. The binding molecule, for example the single variable domain, or the single domain antibody, or the polypeptide, can be recovered by conventional protein purification procedures, including e.g. protein A-Sepharose, hydroxylapatite chromatography, gel electrophoresis, dialysis, or affinity chromatography, using e.g. strepavidin/biotin (see e.g. Low et al., 2007, J. Chromatography B, 848:48-63; Shukla et al., 2007, J. Chromatography B, 848:28-39).

The present invention also provides for an in vitro method for detecting plasmin-cleaved VWF in a subject. The method preferably comprises the steps of: i); contacting a sample from the subject with a binding molecule as described herein; and, ii) analysing the product of step i) to determine the presence and/or the amount of plasmin-cleaved VWF contained in the sample. The method can further include the step of providing a sample from the subject.

The method according to the invention may also be used for determining the activity of a therapeutic agent regulating plasmin-mediated cleavage of VWF in a subject. The therapeutic agent may regulating plasmin-mediated cleavage of VWF by, for example, inducing it or inhibiting it, increasing or decreasing the amount of plasmin in the subject, activating or deactivating the plasmin in the subject, or increasing or decreasing the amount of plasminogen in the subject. The regulation may be through any mechanism known to the person skilled in the art.

In another aspect, the invention provides of a method for determining the occurrence of a vascular event in a subject. The method comprises the method comprises assaying a sample from the subject with a binding molecule of the present invention to determine a concentration of plasmin-cleaved VWF contained in the sample; and determining the occurrence of a vascular event in the subject if the concentration of plasmin-cleaved VWF is, at least 1 ng/ml, 2 ng/ml, 3 ng/ml, 4 ng/ml, 5 ng/ml, 6 ng/ml. 7 ng/ml, 8 ng/ml, 9 ng/ml, 10 ng/mL, 15 ng/ml, 20 ng/ml, for example between 4-10 ng/mL. The assaying step may be performed according to the method of the present invention as described herein for detecting VWF in a subject.

In one embodiment, the method for determining the occurrence the occurrence of a vascular event in a subject comprises detecting the presence of a plasmin-cleaved VWF using a single variable domain that comprises the combination of CDR sequences SEQ ID NO: 1, SEQ ID NO: 2, and SEQ ID NO: 3 wherein the presence in the subject's sample of the plasmin-cleaved VWF at a concentration of at least 8 ng/ml is indicative of the occurrence of a vascular event.

In one embodiment, the method for determining the occurrence the occurrence of a vascular event in a subject comprises detecting the presence of a plasmin-cleaved VWF using a single variable domain that comprises the combination of CDR sequences SEQ ID NO: 4, SEQ ID NO: 5, and SEQ ID NO: 6 wherein the presence in the subject's sample of the plasmin-cleaved VWF at a concentration of at least 4 ng/ml is indicative of the occurrence of a vascular event.

A vascular disorder is an event, disease or disorder that is either a thrombotic occlusion, and/or that occurs after damaging or activation of the endothelium. Damaging or activation or the endothelium leads to VWF secretion which can contribute to a thrombotic event or increases the adhesiveness of the endothelium and contributes to extravasation of inflammatory cells which can cause thrombo-inflammatory conditions. A thrombotic occlusion occurs for example when a clot forms and lodges withing a blood vessel. The blockage can fully or partially block the blood vessel causing a vascular disorder. Thrombin generation refers to the activation, expression or up-regulation of thrombin, which is involved in clot formation and an inductor of platelet activation. Activation of platelets via thrombin or other pathways, can lead to VWF release by the activated platelets.

In one embodiments the vascular event is a disease or condition selected from the group consisting of: acquired or hereditary thrombotic thrombocytopenia purpura (TTP) complement-mediated thrombotic microangiopathy, haemolytic uremic syndrome, antiphospholipid antibody syndrome, non-occlusive thrombosis, the formation of an occlusive thrombus, arterial thrombus formation, acute coronary occlusion, peripheral arterial occlusive disease, restenosis and disorders arising from coronary by-pass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, transient cerebral ischemic attack, unstable or stable angina pectoris, cerebral infarction, HELLP syndrome, carotid endarterectomy, carotid artery stenosis, critical limb ischemia, cardioembolism, peripheral vascular disease, restenosis, sickle cell disease myocardial infarct, a bleeding episode, acute ischemic stroke, pulmonary embolism, Chronic thromboembolic pulmonary hypertension, SEPSIS, COVID-19 and transplant associated thrombotic microangiopathy, medical device-associated thrombosis, acute- or chronic liver disease.

Preferably, the vascular event is acquired or hereditary thrombotic thrombocytopenia purpura (TTP) complement-mediated thrombotic microangiopathy.

In a method according to the invention, the sample may be any appropriate sample known to the person skilled in the art, preferably selected from the group consisting of urine, blood, serum, and blood plasma.

In all embodiments of the invention, a subject is a human or an animal, for example a human, a rat, a mice, or a guinea pig. Preferably, the subject is a human. In certain embodiments the subject is a human female.

The present invention also provides a kit comprising a) an element comprising a binding molecule of the present invention; and b) means for detecting the binding of the element to plasmin-cleaved VWF.

The means for detecting may take any one of a variety of forms, and include detectable labels that are associated with and/or linked to the element. Detectable labels that are associated with and/or attached to a secondary binding ligand are also contemplated. Exemplary secondary ligands are those secondary antibodies that have binding affinity for the binding molecule. Optionally, the kit also comprise means for collecting a sample from a subject. For example he kit may comprise means for collecting a blood sample or a urine sample from a human patient.

All patent and literature references cited in the present specification are hereby incorporated by reference in their entirety.

Whilst the present invention has been described and illustrated with reference to particular embodiments, it will be appreciated by those of ordinary skill in the art that the invention lends itself to many different variations not specifically illustrated herein. By way of example only, the present invention is further described by the following examples which should not be construed as limiting the scope of the invention.

### DESCRIPTION OF THE FIGURES

FIG. 1: Purified plasma-derived VWF before and after plasmin cleavage. Western blot of purified VWF and plasmin-cleaved VWF (cVWF) without (left panel) and with reduction (right panel).

FIG. 2: PPACK inhibits plasmin activity. Plasmin activity was monitored with a fluorogenic substrate in absence or presence of PPACK (75 ug/mL plasmin, 217.8 M PPACK).

FIG. 3: Time-dependent VWF cleavage by plasmin during microthrombus breakdown. (A) Time course of sampling during microthrombus breakdown by plasmin (75 μg/mL). Plasmin activity was blocked with PPACK, cells removed by centrifugation and supernatants analysed by Western blotting. (B) Western blot under non reducing conditions (C) Western blot under reducing conditions. Data represent three independently executed experiments. Plm=plasmin

FIG. 4: Multimer analysis of cVWF. VWF multimers were separated on 2% SDS-agarose gel and visualized as previously described. 6 NPP=normal pooled plasma, HMWM=high molecular weight multimers, LMWM=low molecular weight multimers

FIG. 5: Screening results from VHH selection against cVWF. A) Screening of VHH binding to immobilized biotinylated intact VWF (Biotin-VWF) and plasmin-cleaved VWF (cVWF; Biotin-cVWF). B) Capturing of VWF and cVWF in buffer by immobilized anti-VWF VHH B9.

FIG. 6: ELISA with VWF and plasmin-cleaved VWF in buffer. The ELISA was performed with cVWF-specific capture VHH #1 (A), #2 (B) or a non-binding control VHH (C: Negative Control).

FIG. 7: The cVWF ELISA specifically recognizes plasmin-cleaved cVWF, but not ADAMTS-13-cleaved cVWF in buffer. The cVWF ELISA was performed with cVWF capture VHH #1 (A) or #2 (B).

FIG. 8: Formation and detection of plasmin-cleaved VWF during the thrombolysis of platelet-agglutinates. A) Plasmin mediated thrombolysis of VWF-platelets agglutinates. B) Western blot of thrombolysis supernatant samples under reducing conditions, cVWF levels (as measured by cVWF ELISA with capture VHH #1 or #2; (C) or (D) respectively) in thrombolysis supernatant samples as a fraction of total VWF antigen (as measured by VWF antigen ELISA).

FIG. 9: Detection of plasmin-cleaved VWF in citrated plasma. Detection of plasmin-cleaved VWF (cVWF) by the cVWF ELISA in spiked citrated plasma. The cVWF ELISA was performed with cVWF capture VHH #1 (A), #2 (B) or a non-binding VHH (C: Negative Control). D) Reducing western blot of eluted (c) VWF from the cVWF ELISA (immobilized Capture VHH #2).

FIG. 10: cVWF Assay. (A) cVWF in buffer or (B) citrated normal pooled plasma in the absence or presence of ristocetin (600 μg/mL). (C) cVWF or ADAMTS13-cleaved VWF in buffer or normal pooled citrated plasma. Data are expressed as means+/−SD for three independently executed experiments.

FIG. 11: Characterization of a binding site in cVWF that enables VHH capture. Conditioned cell culture supernatants containing C-terminally truncated VWF variants were incubated with plasmin (150 μg/mL) or vehicle and analysed by (A) Western blotting and by ELISA. (B) VHH #2 was immobilized and captured VWF variants were detected with a polyclonal antibody. (C) Western blot of captured VWF products, spiked in buffer or normal pooled plasma. Data represent three independently executed experiments. Bar graphs show means+/−SD. M=marker; Neg. Sup=Negative control supernatant; Plm=plasmin.

FIG. 12: cVWF release from microthrombi. Preformed microthrombi were separated from soluble VWF by centrifugation and exposed to plasmin (75 μg/mL) or buffer. Platelet complexes were removed by a second centrifugation step and supernatants analysed by (A) Western blotting and (B) ELISA. Data represent three independently executed experiments. Bar graphs show means+/−SD. No Plt=No platelets. Plm=plasmin.

FIG. 13: PPACK protects VWF from cleavage by plasmin. VWF was exposed to plasmin in the presence or absence of PPACK, cVWF formation was determined by ELISA. Conditions were selected to reflect those of FIG. 14 (50 μg/mL plasmin, 50 UM PPACK) to ensure that samples that were collected from the flow system did not progressively form more cVWF because of incomplete plasmin inhibition. (A-I) Reagents were added in a varying sequence. The incubation time was 810 seconds (duration of the flow experiments in FIG. 14)

FIG. 14: cVWF formation under flow. Washed platelets were perfused over histamine stimulated HUVECs. After being stable for 10 minutes, HUVEC-bound platelet-covered VWF strings were exposed to plasmin (50 μg/mL) or vehicle control under flow. Samples were collected into PPACK and analysed for VWF antigen and cVWF levels by ELISA. Data represent five independently executed experiments, and are shown as means+/−SD.

FIG. 15: cVWF formation during thrombotic microangiopathy attacks in TTP patients. (A-C) VWE antigen levels, cVWF levels and fraction of VWF in a plasmin-cleaved state in healthy controls, TTP patients in remission and during acute attacks. Data are shown as scatterplots with medians. Data were analysed with Kruskall-Wallis test followed by Dunn's multiple comparisons test. *=P<. 05. ****=P<. 0001, ns=non-significant. (D-F) Correlations between (D) PAP complex levels and platelet counts, (E) cVWF levels and platelet counts, (F) cVWF levels and PAP complex levels. (G-H) Correlations between (G) VWF antigen and cVWF levels, (H) VWF antigen levels and platelet counts, (I) VWF antigen levels and PAP complex levels. Correlations were computed by Pearson's correlation coefficients. Healthy=Healthy controls.

FIG. 16: Therapeutic plasminogen activation accelerates cVWF formation. Levels of (A) VWF antigen, (B) cVWF and (C) fraction of circulating VWF that exists in a plasmin-cleaved state after Microlyse treatment (blue squares) or saline treatment (red circles) in Adamts13−/− mice, challenged by administration of rhVWF. Data are displayed as scatterplots with medians. Results were analysed by two-way ANOVA followed by Sidak's post-hoc test. *=P<. 05, **=P<005, ***=P<. 005, ****=P<. 0001, ns=non-significant.

FIG. 17: cVWF is not detectable 24 hours after Microlyse treatment in Adamts13−/− mice, challenged by administration of rhVWF. Levels of (A) VWF antigen, (B) cVWF and (C) fraction of circulating VWF that exists in a plasmin-cleaved state after saline administration (red squares) or Microlyse treatment (blue triangles). Results were analyzed by Kruskall-Wallis test followed by Dunn's multiple comparisons test. *=P<05, **=P<005, ns=non-significant.

### EXAMPLES

### Material and Methods

### Reagents

NaCl, Tween-20, Tween-80, triethylamine, benzamidine hydrochloride, bovine serum albumin (BSA), GelRed, tranexamic acid, histamine, glycine, chloramphenicol, lysozyme, sodium dodecyl sulfate (SDS), bromophenol blue, DL-Dithiothreitol (DTT) and sucrose were obtained from Sigma Aldrich, Saint Louis, Missouri, USA. PCR cleanup kit and nucleospin plasmid kit were from Macherey & Nagel, Düren, Germany. Clonejet (pJET1.2 vector kit), TOP10 E. coli, BL21 pLysS E. coli, 293-fectin, lipofectamine-2000, lipofectamine-3000, HEK293-F cells, Optimem, DMEM (with high glucose, HEPES and L-glutamine), Gentamicin/Amphotericin B mix, Penicillin/Streptomycin mix, Freestyle 293 Expression medium, isopropyl β-D-1-thiogalactopyranoside (IPTG), 4-12% Bis Tris-Page gel, MOPS buffer, methanol, Page Blue protein staining solution and neutravidin were from Thermo Fisher, Waltham, Massachusetts, USA. Glucose and 4-(2-hydroxyethyl)-1-piperazineethanesulfonic acid (HEPES) were from VWR Avantor Life Science, Radnor, Pennsylvania, USA. Endothelial Growth Medium-2 (EGM-2, C-22011) and SupplementMix (C-39216) were from PromoCell, Heidelberg, Germany. Polyethylene glycol 6000 (PEG 6000), imidazole, sodium-acetate, H2SO4, MgSO4, glutaraldehyde, sodium citrate, ethanol and the Immobilon-FL membrane were obtained from Merck, Darmstadt, Germany. Fetal Calf Serum was from Lonza, Basel, Switzerland. Ampicillin was from Carl-Roth, Karlsruhe, Germany. The phusion PCR polymerase kit and the digestion enzymes EcoRV, NotI, BamHI, Sbfl, PspXI and Nhel were from New England Biolabs, Ipswich, Massachusetts, USA. Yeast-Tryptone agar, 2×Yeast-Tryptone medium and human serum albumin were from MP Biomedicals, Irvine, California, USA. Tris and DNAse I were from Roche, Basel, Switzerland. Maxisorp plates were from Nunc, Thermo Fisher, Waltham, Massachusetts, USA. Hydrogen chloride (HCl) was from Fluka Honeywell, Charlotte, North Carolina, USA. Ficoll was from G&E Healthcare, Chicago, Illinois, USA. Plasma derived VWF was purified via gel filtration from Haemate-P, CSL Behring, King of Prussia, Pennsylvania, USA. PGI2 was from Sanbio, Uden, the Netherlands. FPR-Chloromethylketone (PPACK) was from Enzyme Research Laboratories, South Bend, Indiana, USA. Plasmin substrate I-1390 was from Bachem, Bubendorf, Switzerland.

3,3′,5,5′-Tetramethylbenzidine (TMB) was from Tebubio, Heerhugowaard, Netherlands. Glycerol and MgCl2 was from Honeywell Riedel-de-Haën, Seelze, Germany. RGDW peptide was synthesized by NKI, Amsterdam, the Netherlands. Iloprost was from Bayer, Leverkusen, Germany. Ristocetin was from Biopool International, Ventura, California, USA. Recombinant VWF was from Takeda (Vienna, Austria). Streptokinase was from CSL-Behring, King of Prussia, Pennsylvania, USA. Odyssey blocking reagent was from LI-COR Biosciences, Lincoln, Nebraska, USA.

Antibodies: HRP-rabbit anti-camelid VHH (A02014, GenScript, Piscataway, New Jersey, USA), rabbit polyclonal anti-human VWF (A0082, Agilent Dako, Santa Clara, California, USA) for Western blot and VWF antigen ELISA. HRP-conjugated rabbit anti-human VWF detection polyclonal antibody (P0226, Agilent Technologies, Santa Clara, California, USA). IRDye 680LT goat anti-rabbit lgG (P/N 926-68021, LI-COR Biosciences, Lincoln, Nebraska, USA) for Western blot, Poly-HRP-conjugated streptavidin (M2051, Streptavidin poly HRP, Sanquin, Amsterdam, Netherlands).

### SDS PAGE and Western Blotting

Samples were diluted in 3× sample buffer (30% glycerol, 0.18 M Tris-HCl, 6% SDS, Bromophenol blue, with or without 25 mM DTT for reduction), after which they were heated for 10 min at 95° C. Samples were separated on 4-12% Bis-Tris gel in MOPS-SDS running buffer. Protein bands were blotted onto an immobilon-FL membrane at 125V for 90 minutes in blotting buffer (25 mM Tris, 192 mM Glycine, 20% v/v ethanol). The blots were blocked overnight at 4° C. in Odyssey blocking reagent diluted 1:1 in tris-buffered saline (TBS: 25 mM Tris, 150 mM NaCl pH 7.4). Blots were incubated with a polyclonal rabbit anti-VWF antibody (DAKO; 1 μg/mL in odyssey blocking reagent) for 2 hours at room temperature while mixing. Membranes were washed with 0.05% v/v Tween 20 in TBS (TBST) and incubated with goat-anti-rabbit-Alexa 680 (Thermo Fisher; 1:5000 in odyssey blocking reagent). Membranes were washed with TBST and TBS, where after they were analyzed on a near infrared Odyssey scanner (LI-COR).

### Plasmin or ADAMTS13 Cleaved VWF

Purified VWF (44.6 μg/mL) and 0.6 mg/mL Ristocetin were incubated with plasmin (143 μg/mL) in 2% BSA-PBS at 37° C. Reactions were stopped by the addition of 250 μM PPACK. Alternatively, purified VWF and (1.5 M Urea) was incubated with ADAMTS13 (1 μg/mL; preactivated in 10 mM CaCl₂), 150 mM NaCl, 0.05% Tween-20 pH 7.5) in HEPES buffered saline (HBS; 10 mM HEPES, 150 mM NaCl, pH 7.4). Reactions were stopped by the addition of 10 mM EDTA.

### Plasmin Activity Assay

Plasmin activity in the presence or absence of PPACK was monitored with a fluorogenic plasmin substrate. Plasminogen (1.22 mg/mL) was incubated with 2000 IU/mL streptokinase for 15 min at 37° C. to form plasmin. Plasmin (75 μg/mL) was subsequently incubated with PPACK (217.8 UM) or vehicle in 0.2% (w/v) HBS-BSA for five minutes at 37° C. Next, plasmin substrate I-1390 was added (0.5 mM) and fluorescence measured at 37° C. with an extinction of 380 nm and emission of 460 nm for 60 min.

### Blood Collection and Washed Platelet Preparation

Whole blood from healthy donors was obtained under approval by the Medical Ethical Committee of the University Medical Center Utrecht. Blood samples were collected in sodium citrated collection tubes on the day of experiments and centrifuged at 160×g for 15 min to obtain platelet-rich plasma (PRP). Platelet counts were determined by CELL-DYN Emerald Hematology Analyzer (Abbott Laboratories, Chicago, Illinois, USA). Prior to further centrifugation at 400×g for 15 min for the separation of platelet-poor plasma (PPP), citrate-dextrose solution (ACD; 8.5 mM tri-sodium citrate, 7.1 mM citric acid, 11.1 mM D-glucose) was added to the undiluted PRP. PPP was removed and pellets were resuspended back to their initial volume using HEPES Tyrode buffer (HT buffer; 145 mM NaCl, 5 mM KCl, 0.5 mM Na₂HPO₄, 1 mM MgSO₄, 10 mM HEPES, 5.55 mM D-glucose, pH 6.5). PGI2 (10 ng/ml final concentration) was added to platelet suspensions prior to another centrifugation step at 400 xg for 15 min. Supernatants were removed and the pellets were resuspended in HT buffer (pH 7.3); platelet counts were adjusted to 200×10⁹/L.

### Example 1—VWF Cleavage During Destruction of Microthrombi by Plasmin

<Purification of VWF from Haemate-P>

Lyophilized Haemate-P (plasma derived FactorVIII/VWF concentrate from CSL Behring) was dissolved with distilled water according to manufacturer instructions. A 200 pg HiLoad 26/600 Superdex column (G&E Healthcare, Chicago, Illinois, USA) was preequilibrated with gel filtration buffer (50 mM Tris, 150 mM NaCl, 5 mM Tri-sodium citrate, pH 7.4), whereafter the Haemate-P was applied to the column to separate VWF (multimeric form of 250 kDa monomers) and FVIII (330 kDa) based upon their size, and pooled and buffer exchanged against phosphate buffer saline (PBS; 21 mM Na₂HPO₄, 2.8 mM Na₂HPO₄, 140 mM NaCl, pH 7.4) via a HiTrap desalting column (G&E Healthcare, Chicago, Illinois, USA). The VWF containing fractions were confirmed by western blot and pooled. Protein concentration was determined via absorbance at 280 nm and the extinction coefficient was determined via ProtParam. Purity and degradation were assessed via 4-12% Bis Tris-Page gel and Coomassie blue staining. Protein containing fractions were pooled again, whereafter the VWF concentration was confirmed a VWF antigen ELISA as previously published (PMID: 24449821).

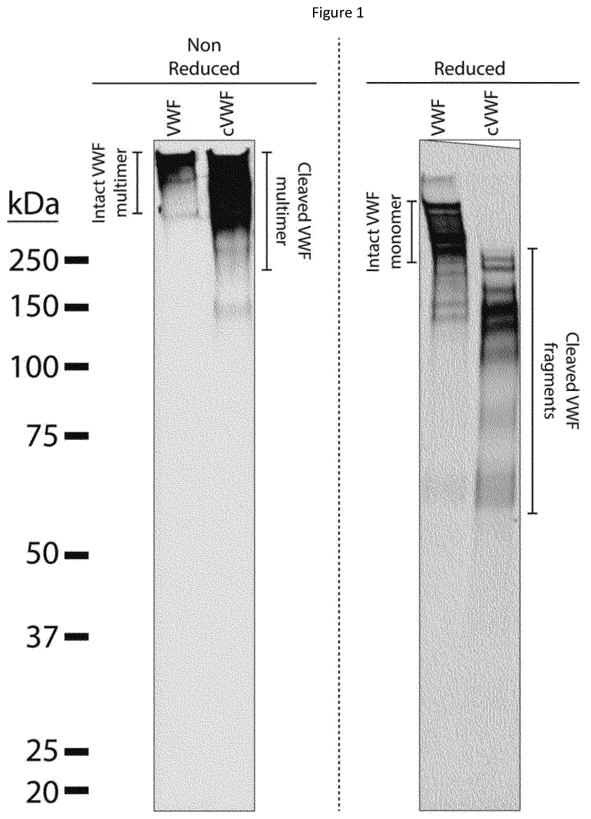

The isolated VWF exists as a covalently bound multimer (FIG. 1; VWF non reduced) that consists of ˜250 kDA monomers (FIG. 1: VWF reduced).

### <Generation of Purified Plasmin-Cleaved VWF>

Plasma purified plasminogen (purified as described previously PMID: 27130860) was activated by streptokinase for 15 minutes at 37° C. Purified VWF (VWF levels quantified via ELISA as previously described) was incubated with 0.6 mg/mL Ristocetin (American Biochemical & Pharmaceuticals Ltd.) and 143 μg/mL plasmin for 60 minutes, where after plasmin was inhibited via the additions of 200 mM Benzamidine (Sigma-Aldrich) and 200 mM tranexamic acid (Sigma-Aldrich). The cleaved VWF (cVWF) was separated from plasmin via the 200 pg HiLoad 26/600 Superdex column as described above but in the presence of 20 mM Benzamidine and 200 mM tranexamic acid. The inhibitors and trace amounts of plasmin were further removed via the HiTrap desalting column (G&E Healthcare; precalibrated in phosphate buffer saline (PBS; 21 mM, Na₂HPO₄, 2.8 mM NaH₂HPO₄, 140 mM NaCl, pH 7.4) and a lysine-Sepharose 4B column (G&E Healthcare). Protein containing fractions were identified via absorbance at 280 nm and purity and degradation were assessed via 4-12% Bis Tris-Page gel and western blotting. The plasmin-cleaved VWF (cVWF) exists mainly as a covalent bound-multimer (FIG. 1; cVWF non reduced) that consists of various sized fragments (FIG. 1: cVWF reduced).

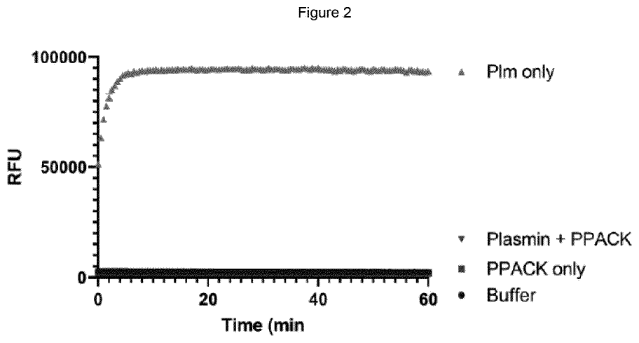

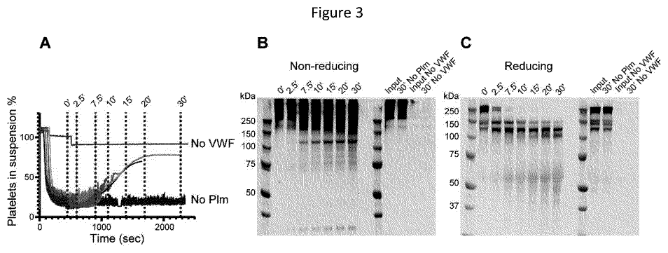

In another test, we generated microthrombi (platelet agglutinates) in vitro by incubating washed platelets with ristocetin-activated VWF. Plasminogen was purified from human citrated plasma as described previously. Plasminogen (1.22 mg/mL) was activated with streptokinase (2000 IU/mL) for 15 min at 37° C. Six min after triggering of platelet agglutination with ristocetin (800 μg/mL), streptokinase-activated plasminogen (50 μL, final concentration 75 μg/mL) was added. Plasmin was inactivated at indicated time points by the addition of PPACK (5 μL, 12.5 mM working stock, 217.8 UM final concentration). Whole samples were spun down for 15 min at 400×g and supernatants were collected for SDS PAGE and Western blot analysis. The results are shown in FIG. 2. Microthrombus formation does not occur in the absence of VWF, and formed microthrombi do not disassemble in the absence of plasmin, indicating that these microthrombi are stable and VWF-dependent (FIG. 3A).

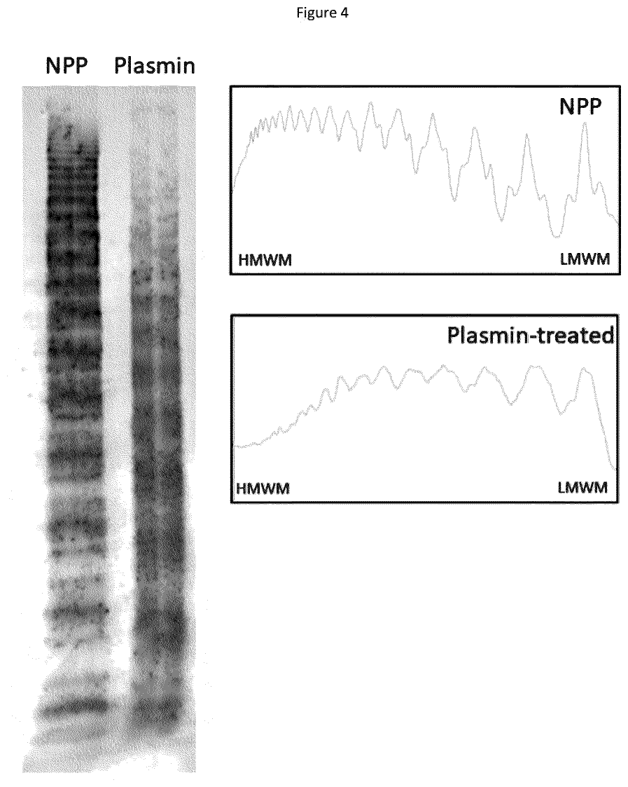

VWF migrates as a large smear with a mass >250 kDa (the size of VWF monomer) on SDSPAGE under non-reducing conditions, owing to its disulfide-linked polymeric state. Exposure to plasmin leads to a shift in this migration pattern with part of the smear being between 250-150 kDa (smaller than VWF monomer). Additional smaller separate fragments appear at ˜120 kDa and ˜30 kDa (FIG. 3B). When analysed under reducing conditions, the 250 kDa VWF monomer is cleaved within minutes, indicating that each monomer has been cleaved at least once, without complete depolymerization (FIG. 3C). Cleavage products appear at 140 and 120 kDa, and later at 110 kDa. There is progressive formation of a ˜70 kDa product. This indicates that the predominant cleavage product is still polymeric and disulfide-linked. A multimer analysis shows loss of larger multimers (FIG. 4).

### Example 2—cVWF ELISA Development

The first step is to select VHH specific for plasmin-cleaved VWF.

### <Llama Immunization and Bacteriophage Library Preparation>

Llama glama received four subcutaneous injections with plasma-derived VWF during a 4-week period. Twenty-four hours after the last injection, venous blood was collected from which peripheral blood lymphocytes were isolated by Ficoll gradient. Total RNA was isolated and transcribed into cDNA from which VHH genetic libraries in the E. coli TG1 strain were created, where a transformation efficiency over 107 was achieved for both phage display libraries. Phages were produced from the VHH-libraries overnight and isolated via polyethylene glycol (PEG; Mr=6000) precipitation (20% m/v PEG6000 in 2.5M NaCl) as previously described (PMID: 27130860, 23349032).

### <Phage Display Against Biotinylated Plasmin-Cleaved VWF>

The purified VWF and cVWF were biotinylated with NHS-LC-Biotin (Thermo Fisher) according to manufacturer instructions. The biotinylated (c) VWF (biotin-VWF and biotin-cVWF respectively) were buffer exchanged against PBS via a Zeba desalting column (Thermo Fisher) to remove excess uncoupled NHS-LC-Biotin.

Maxisorp 96-well plates were coated with Neutravidin (5 μg/mL in PBS) overnight at 4° C. Coated plates were blocked with 2% w/v bovine serum albumin (BSA) in PBS (BSA-PBS; 200 μl/well) for 1 hour at room temperature, while shaking. Biotin-cVWF (1 μg/mL in 0.5 w/v BSA-PBS; 50 μl/well) was added to the blocked plates and incubated for 1 hour at room temperature, while shaking. Isolated phages were blocked in 2% BSA-PBS for 1 hour at room temperature). Wells were rinsed with 200 μl/well 0.05% v/v Tween-20 in PBS (PBS-T). Preblocked phages were added to the plate (10 μL phages/well) and incubated for 2 hours at room temperature. Wells were rinsed 15 times with PBS-T, where after bound phages were eluted by the addition of 50 μL triethylamine (0.1M in H₂O). Eluted phages were transferred to sterile Eppendorf tubes containing 100 μL 1M Tris-HCl (pH 7.5). Isolated phages were added to exponentially growing E. coli (TG1) for infection at 37° C. for 30 minutes without shaking. Infected bacteria were added to 2×YT media containing (2% w/V) glucose and ampicillin (100 μg/mL) and incubated overnight at 37° C., while shaking. From the 2×YT culture, new phages were produced for the 2nd round of selection.

For the 2nd round of selection, the phages were produced from the outcome of the 1st round of selection and were first preincubated with intact purified VWF, to increase specificity against cVWF in the 2nd round of selection. Hereto phages were blocked in 2% BSA-PBS for 1 hour at room temperature. Hereafter, purified non-biotinylated VWF was added to the preblocked phages for 1 hour at room temperature, after which the preblocked phages were added to plate for selection against biotinylated-cVWF as described above. Wells were rinsed 15 times with PBS-T, whereafter bound phages were eluted by the addition of 50 μl triethylamine (0.1M in H₂O). Eluted phages were transferred to sterile Eppendorf tubes containing 100 μl 1M Tris-HCl (pH 7.5). Isolated phages were added to exponentially growing E. coli (TG1) for infection at 37° C. for 30 minutes without shaking. 90 μl of the infected bacteria were plated on Yeast Tryptone (YT)-agar plates containing (2% w/v) glucose and ampicillin (100 μg/mL) and incubated overnight at 37° C. The remained of the infected bacteria were added to 2×YT media containing (2% w/v) glucose and ampicillin (100 μg/mL) and incubated overnight at 37° C., while shaking. Colonies were picked and grown overnight at 37° C. as individual 100 μL cultures in 2× Yeast Tryptone-media containing (2% w/v) glucose and ampicillin (100 μg/mL), while shaking.

### <Screening of VHH Specific Against Plasmin-Cleaved VWF>

10 μL of the individual overnight VHH cultures were added to 90 μL of 2× Yeast Tryptone-media containing (2% w/v) glucose and ampicillin (100 μg/mL) in 2 mL 96-well sterile assay plates and grown for 4 hours at 37° C., while shaking. Plates were centrifugated at 2000×g for 10 minutes, whereafter the supernatant was removed. Pellets were resuspended in 600 μL/well of 2× Yeast Tryptone-media containing ampicillin (100 μg/mL) and isopropyl β-d-1-thiogalactopyranoside (IPTG; 1 mM) and plates were cultivated overnight at 37° C., while shaking. Plates were centrifugated at 2000×g for 10 minutes whereafter the supernatant (containing the produced VHH's) were transferred to an 0.8 mL 96-well plate and stored at −20° C. until further use.

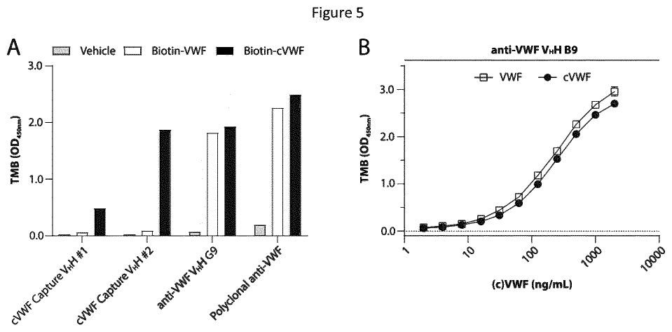

Biotin-VWF or biotin-cVWF was immobilized via neutravidin onto 96-well Maxisorp plates as described above. VHH containing supernatants were mixed 1:1 with 2% BSA-PBS after which 50 μL/well was added to the coated plates and incubated at room temperature, while shaking. Plates were washed 3× with 150 μl/well PBS-T, after which bound VHH's were detected by a cocktail of HRP-labelled anti-VHH IgG (Genscript; 1 μg/mL in 2% w/v BSA-PBS). As a positive control to confirm equal loading of biotin-VWF or biotin-cVWF, captured (c) VWF was detected via a polyclonal IgG HRP-labelled rabbit-anti VWF antibody (DAKO; 1:1000 in 2% w/v BSA-PBS; 50 μL/well). This was incubated for 1 hour at room temperature while shaking. Plates were washed 3× with 150 μL/well PBS-T, after which 100 UL of 3,3′,5,5′-Tetramethylbenzidine (TMB; Tebu-bio) was added. Substrate development was allowed for a maximum of 30 minutes where after the reaction was stopped via the addition of 50 μL 0.3M H₂SO₄. Absorbance was measured at 450 nm and results were analysed by GraphPad Prism 9.2. From clones of interest, plasmid DNA was isolated using the Nucleospin plasmid kit (Macherey-Nagel) according to manufacturer instructions. VHH sequences were determined via Sanger sequencing (Primer: GAGCGGATAACAATTTCACACAGG). Sequencing data was transformed in amino acid sequences for comparison via Clonemanager 7.10. Results are shown in FIG. 5A

### <VHH Production and Purification>

### Shaker Bottle Production for Anti-VWF VHH B9

The clone for anti-VWF VHH B9 was grown at 37° C. overnight in 2× Yeast Tryptone-media containing (2% w/v) glucose and ampicillin (100 μg/mL), while shaking. The culture was diluted 1:100 in 500 mL 2× Yeast Tryptone-media containing ampicillin (100 μg/mL). The culture was grown in baffled shaker-flask 37° C. for 3 hours while shaking. When the culture reached an OD600 of ˜0.5, protein production was induced via the addition of IPTG (1 mM), whereafter production was continued overnight at 24° C., while shaking. The culture was centrifugated at 5000×g for 15 minutes after which the supernatant was removed. Pellets were resuspended in PBS containing the Complete inhibitor mix (Roche; no EDTA) and frozen at −20° C. until further use. Samples were thawed at 37° C. and centrifugated at 10.000×g for 30 minutes. Supernatant was transferred to new tubes, whereafter TALON Superflow beads (Cytiva) were added and incubated for 2 hours at room temperature, while mixing. Beads were centrifugated at 1000×g for 10 minutes, whereafter the supernatant was removed. Beads were washed 3× with PBS with intermitted centrifugation steps, whereafter the beads were loaded into a PD-10 column (Cytiva). The columns were washed with PBS until no protein was detected anymore in the flowthrough. The VHH was eluted by applying 150 mM Imidazole in PBS to the column. Protein fractions were pooled and dialyzed against PBS overnight at 4° C. The purified VHH was biotinylated with NHS-LC-Biotin as described above.

### Fermenter Production for Capture VHH's

The VHH sequences for the capture VHH's were codon-optimized DNA sequence for expression in E. coli via the online tool of IDT. At the 5′ end of this sequence, a BamHI digestion site was added and at the 3′ end, a NotI digestion site was added. The constructs were ordered as a double stranded DNA fragment from IDT. The double-stranded DNA fragments were ligated into the pJET1.2 cloning vector according to manufacturer's instructions. The vectors were digested by BamHI and NotI and the inserts were ligated into a modified pET32a+ protein expression vector (Merck-Millipore). This vector encodes a N-terminal PelB leader sequence, His⁶ tag and a TEV protease cleavage site. At the C-terminus, the VHH sequence is followed by a Myc-tag and a stop codon. The expression vectors were transformed into BL21 pLysS E. coli and grown in 2× YT media containing 2% w/v glucose, 100 μg/mL ampicillin and 50 μg/mL chloramphenicol. Hereafter a 5 L fermenter containing ZYP-5052 autoinduction media (without trace metals (PMID: 15915565) was inoculated and grown for 3 hours at 37° C. with 70% dissolved O₂. Hereafter, the temperature was lowered to 24° C. and production continued overnight. Bacteria were pelleted at 5000×g for 15 minutes and subsequently resuspended in binding buffer (25 mM HEPES, 500 mM NaCl, pH 7.8) together with 1 mM MgCl₂ and 1 μg/mL DNAse I. Bacteria were cracked by three freeze-thaw cycles using liquid nitrogen. Hereafter, samples were treated with lysozyme for 30 minutes at 37° C. prior to centrifugation at 10.000×g for 60 minutes. Supernatant was collected and VHH's were isolated using immobilized metal affinity chromatography. The VHH's were further purified and buffer exchanged by gel filtration over a 75 pg HiLoad 26/600 Superdex column that was prefilled with a 20 mM sodium citrate buffer (7% w/v Sucrose, 0.01% v/v Tween 80). pH of the buffer was adjusted for the theoretical pl of the VHH's (6.8 for capture VHH #1 and 6.4 for capture VHH #2 respectively). Protein concentration were determined by absorption at 280 nm and corrected for the extinction coefficient. Purity and degradation were assessed by Coomassie blue staining after SDS-PAGE. VHH were produced and secreted into the growth media, whereafter VHH s were allowed to bind to captured biotin-cVWF or biotinylated VWF (biotin-VWF) (FIG. 5A). Results from these experiments clearly demonstrate that VHHs can discriminate between plasmin-cleaved VWF and intact VWF. Besides specific cVWF VHH's, several non-selective VHH's were found that could act as a detection VHH for the cVWF ELISA. The non-selective VHH B9 was produced, purified and immobilized to a 96-well Maxisorp plate. The capture ELISA using VHH B9 clearly demonstrates that this VHH does not discriminate between VWF and cVWF (FIG. 5B), making it suitable for application as a detection reagent.

<VWF/cVWF Capture ELISA with Anti-VWF VHH B9>

The non-biotinylated anti-VWF VHH B9 was coated onto a 96/well Maxisorp plate (5 μg/mL in PBS; 50 μL/well) overnight at 4° C. The plate was blocked with 2% w/v BSA-PBS (200 μL/well) for 1 hour at room temperature while shaking. The plate was rinsed 3-times with 0.01% v/v PBS-T whereafter VWF or cVWF diluted in 2% w/v BSA-PBS (50 μL/well) was applied to the plate. Samples were incubated at room temperature for 1 hour, while shaking. The plate was rinsed 3-times with 0.01% v/v PBS-T, where after bound VWF/cVWF was detected via the polyclonal IgG HRP-labelled rabbit-anti VWF antibody (1:1000 in 2% w/v BSA-PBS; 50 μL/well) for 1 hours at room temperature, while shaking. The plate was rinsed 3-times with 0.01% v/v PBS-T. Hereafter the plate developed by the addition of 100 μL of TMB substrate. Substrate development was allowed for a maximum of 30 minutes where after the reaction was stopped via the addition of 50 μL 0.3M H₂SO₄. Absorbance was measured at 450 nm and results were analysed by GraphPad Prism 9.2. Results are shown in FIG. 5B.

<cVWF Capture ELISA>

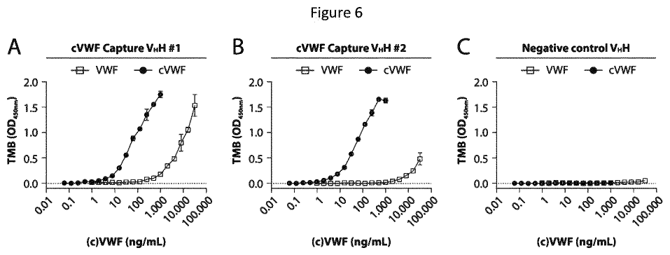

The cVWF capture VHH's were coated onto a 96/well Maxisorp plate (5 μg/mL in PBS; 50 μL/well) overnight at 4° C. Plates were blocked with 2% w/v BSA-PBS (200 μL/well) for 1 hour at room temperature while shaking. Plates were rinsed 3-times with 0.01% v/v PBS-T whereafter diluted samples were applied (See specific sections for sample preparation). Samples were incubated at room temperature for 1 hour, while shaking. Plates were rinsed 3-times with 0.01% v/v PBS-T, where after bound cVWF was detected via either the polyclonal IgG HRP-labelled rabbit-anti VWF antibody (1:1000 in 2% w/v BSA-PBS; 50 μL/well), or via the biotinylated anti-VWF VHH B9 (50 ng/ml in 2% w/v BSA-PBS; 50 μL/well). Detection antibodies were incubated for 1 hours at room temperature, while shaking. Plates were rinsed 3-times with 0.01% v/v PBS-T. For the plates incubated with the biotinylated VWF detection VHH an additional incubation was performed with poly-HRP labelled streptavidin (1:2000 in in 2% w/v BSA-PBS; 50 μL/well) for 1 hour at room temperature, while shaking, followed by 3-times washing with 0.01% v/v PBS-T. Hereafter all plates were developed by the addition of 100 μL of TMB substrate. Substrate development was allowed for a maximum of 30 minutes where after the reaction was stopped via the addition of 50 μL 0.3M H₂SO₄. Absorbance was measured at 450 nm and results were analysed by GraphPad Prism 9.2. Using both cVWF-specific VHH's to capture cVWF and the non-selective VHH B9 for detection, we set out to develop a cVWF-specific capture ELISA. Since purified VWF and cVWF (in absence of bulk proteins) becomes unstable during long-term storage, the Haemate-P VWF/Factor VIII preparation was used as the VWF standard (contains albumin as protectant). For the formation of cVWF, plasmin and Ristocetin were added to the Haemate-P VWF/Factor VIII preparation for 60 minutes, whereafter plasmin activity was inhibited via the addition of PPACK. Capture-VHH s #1 and #2 and a negative control VHH-R2 (SEQ ID NO:9) were directly immobilized on microtiter plates. After washing and blocking, plates were incubated concentration series of VWF and cVWF in buffer. Captured (c) VWF was detected with biotinylated anti-VWF VHH B9, followed by streptavidin-poly HRP. Outcomes from these ELISA's show that the two Capture-VHHs can discriminate between VWF and cVWF (144× and 1250× respectively for Capture-VHHs #1 and #2) (FIG. 6A-B), while the negative control VHH (FIG. 6C) was unable to capture any VWF at the concentrations used. Moreover, both cVWF ELISA setups show a sensitivity of 8 and 4 ng/ml respectively for Capture-VHHs #1 and #2.

### <Characterisation of Capture-VHHs>

Purified VWF and (1.5 M Urea) was incubated with ADAMTS13 (1 μg/mL; preactivated in 10 mM CaCl₂), 150 mM NaCl, 0.05% Tween-20 pH 7.5) in HEPES buffered saline (HBS; 10 mM HEPES, 150 mM NaCl, pH 7.4). Reactions were stopped by the addition of 10 mM EDTA.

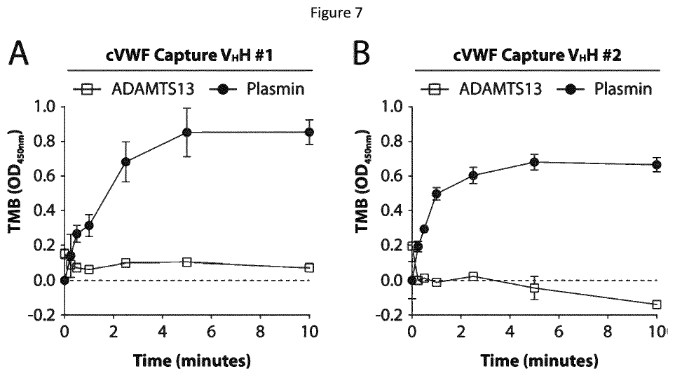

To demonstrate the specificity of our Capture-VHH s for plasmin-cleaved VWF over ADAMTS13 cleaved VWF, VWF was incubated with either plasmin (in the presence of Ristocetin) or ADAMTS13 (in the presence of urea) as described above. At specified timepoints, samples were collected in stop mix (250 UM for plasmin and 10 mM EDTA for ADAMTS13), whereafter cVWF levels were determined by our cVWF ELISA as described. Where plasmin induces a time-dependent formation of cVWF, ADAMTS13 was unable to generate cVWF that could be detected by the cVWF ELISAs (FIG. 7).

Next, we assessed the formation of cVWF during plasmin-mediated thrombolysis of platelet-VWF agglutinates.

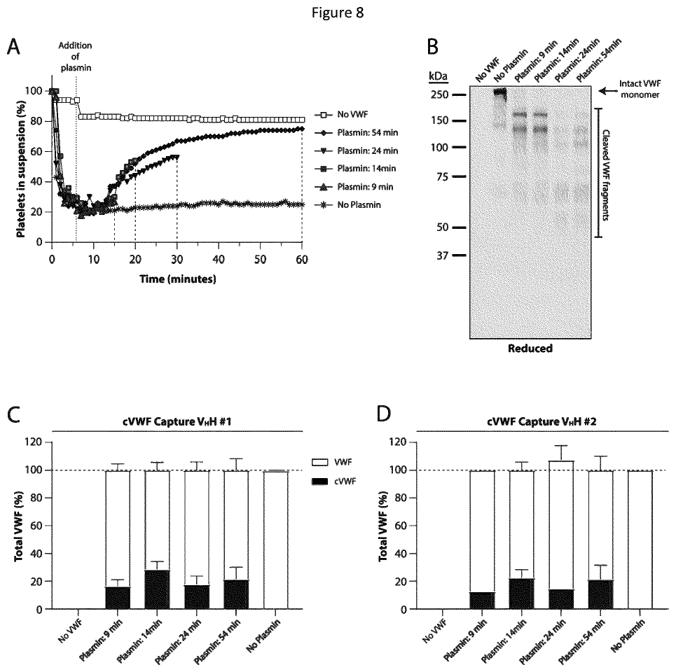

Blood was collected from healthy volunteers who had not been on anticoagulating or antiplatelet therapy medication for at least 10 days before blood withdrawal with informed consent. Blood was anticoagulated with 10% sodium citrate (3.2% w/v). The University Medical Center Utrecht Ethics Committee approved these procedures. Blood platelets were isolated from citrated human whole blood as previously described (PMID: 24449821). Platelets (200.000/μL) were incubated with Haemate-P (66 μg/mL) and the platelet aggregation inhibitors RGDW (Synthesized by Leiden University, the Netherlands; 200 UM) and Iloprost (TEVA B.V.; 0.4 μg/mL) for 15 minutes at 37° C. in a light transmission aggregometer while mixing at 900 rpm. Platelet agglutination was triggered via the addition of Ristocetin (0.8 mg/mL). Upon the addition of Ristocetin, platelet-VWF agglutinates formed, consuming the free platelets from solution (FIG. 8A). After 6 minutes (when platelet-VWF agglutinate formation was stable), plasmin (75 μg/mL) was added and agglutinate lysis was monitored. At specified timepoints samples were removed from the machine and the plasmin activity was inhibited via the addition of 200 UM FPR-chloromethylketone (PPACK; Haemtech). Samples were centrifugated at 500×g and the supernatant was transferred to a new tube. Supernatant samples were diluted in 2% BSA-PBS and applied to the cVWF ELISA. For western blotting, pellet and supernatant samples were diluted in non-reducing sample buffer (30% v/v glycerol, 0.18 M Tris-HCl, 6% v/v SDS, Bromophenol blue) where 25 mM DTT was added if samples were to be analysed under reducing conditions. Samples were heated at 95° C. for minutes prior to being applied to the western blot. Analysis by western blotting shown, that VWF becomes rapidly cleaved upon the addition of plasmin (FIG. 8B). Analysis of VWF antigen levels and cVWF levels show that upon the addition of plasmin, cVWF is formed with a maximum of ˜20% of total VWF antigen (FIG. 8C-D).

Finally, we assessed whether the cVWF ELISA is able to detected plasmin-cleaved VWF in plasma.

Vehicle, Citrated Normal Pooled Plasma (NPP) from healthy volunteers or VWF depleted plasma (ΔVWF plasma; ITK Diagnostics BV) was supplemented with PPACK (250 UM) and cVWF or Vehicle. These samples were further diluted 8-times in 2% BSA-PBS (containing 250 UM PPACK) before being applied to the cVWF ELISA.

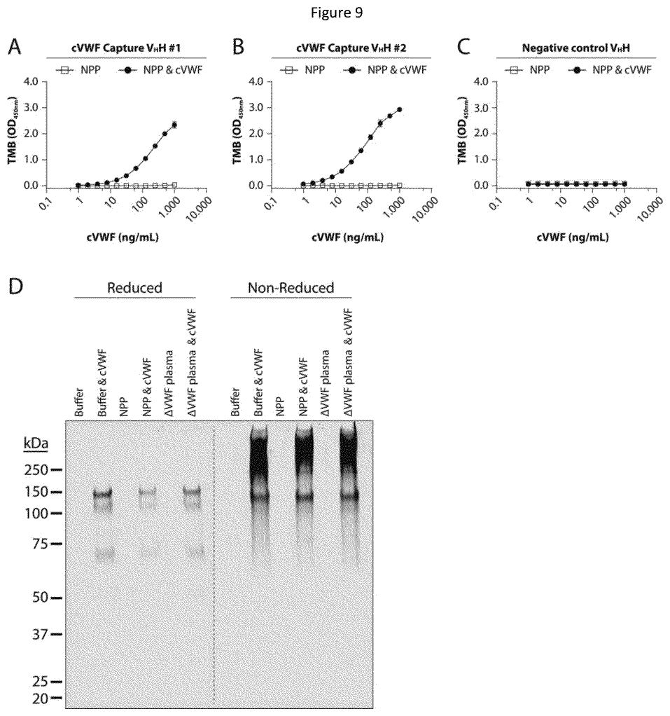

Capture-VHHs #1 and #2 and a negative control VHH were directly immobilized on microtiter plates. After washing and blocking, plates were incubated concentration series of plasmin cVWF or vehicle in normal pooled plasma (NPP) from healthy volunteers. Captured (c) VWF was detected with biotinylated anti-VWF VHH B9, followed by streptavidin-poly HRP. Outcome of these ELISA demonstrate that both cVWF Capture VHH #1 And #2 are able to detect cVWF specifically in plasma with a sensitivity of 8 and 4 ng/ml respectively (FIG. 9A-C). To demonstrate the VWF is truly not captured by Capture-VHHs #2 under these conditions, the experiments was repeated, but rather than applying the biotinylated anti-VWF VHH B9, (non-) reducing sample buffer was applied to microtiter wells to elute the captured (c) VWF from the plates. Furthermore, the experiment was performed in buffer and/or VWF deficient (ΔVWF) plasma rather than NPP as additional controls. The elution samples were analysed by western blotting and demonstrate despite the presence of endogenous VWF in plasma, only plasmin cVWF is capture by Capture-VHHs #2 (FIG. 9D).

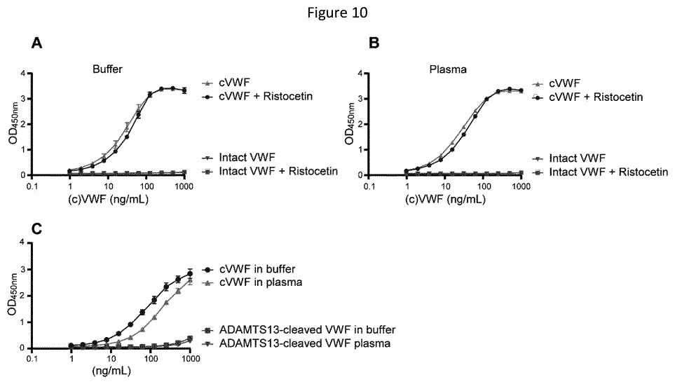

We next set out to further characterize VHH-based bioassay to track plasmin-mediated cleavage of VWF. The assay setup detects cVWF, but not intact VWF, in buffer and in citrated plasma (FIG. 10A,B, respectively) with a lower detection limit of ˜2 ng/ml. The assay is not sensitive to the presence of ristocetin, enabling us to study cVWF release in platelet agglutination experiments. Furthermore, this assay does not recognize the presence of VWF (˜10 μg/mL) or endogenous ADAMTS13-cleaved VWF (7% of the plasma pool by estimation) in citrated plasma. In control experiments, we confirmed the strong preference of this assay for plasmin-cleaved VWF over ADAMTS13-cleaved VWF (FIG. 10C).

The lack of a signal in unspiked plasma means that there is no evidence for the presence of cVWF (i.e. >2 ng/ml) in healthy individuals. This is expected as plasmin-mediated VWF cleavage should only occur during (micro) vascular obstruction, but not under normal physiological conditions.

### Example 3—VHH Binding Site Characterization

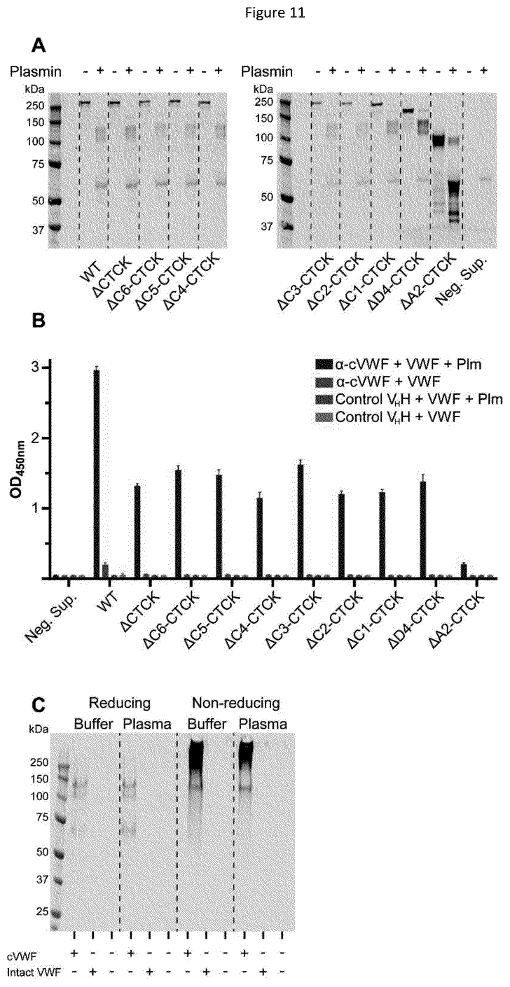

We made recombinant C-terminally truncated constructs of VWF and incubated them with plasmin where indicated (FIG. 11A). Recombinant wild type (WT) VWF forms cleavage products that are similar to those of plasma-derived VWF. When removing the C-terminal cystine-knot domain (ACT/CK), VWF loses its ability to form disulfide-linked multimers. Nevertheless, its cleavage products are very similar. The same holds true for further C-terminal truncation variants beyond ΔCT/CK. All these constructs can be captured by VHH #2, but only in their cleaved state (FIG. 11B). However, truncation of the A2 and A3 domains (ΔA2-CT/CK) results in a strikingly different cleavage pattern and this product is no longer captured (FIG. 11A,B). This indicates that domains A2 and A3 are needed for formation of the plasmin-specific cleavage product that is captured in this ELISA.

Our first experiments indicated that plasmin cleavage of VWF results in partial depolymerisation, as well as formation of soluble fragments (FIG. 3). We next investigated which of these products were captured from spiked buffer or plasma. To that end, we eluted VHH-bound protein from microtiter plates with sample buffer, which we subjected to Western blotting analyses. Under reducing conditions, bands at 140, 120 and 70 kDa are seen (FIG. 11C). Without reduction, the smear >150 kDa is seen, as well as the 120 kDa product. The 30 kDa band present in the cVWF input material (FIG. 3B) is not captured. These cleavage products are not present in unspiked plasma. These experiments indicate that a binding epitope is formed in plasmin-cleaved VWF polymers that is contained in a 120 kDa soluble fragment.

### Example 4—cVWF Release from Microthrombi

Next we explored whether cVWF is released from microthrombi during their degradation. Platelet agglutinates were formed and separated from the reaction mix (containing soluble VWF) using a 18G blunt fill microneedle (Becton Dickinson, USA) attached to a 1 ml syringe (Becton Dickinson, USA), leaving agglutinates intact. Agglutinates were washed once by the addition and subsequent aspiration of 220 μL HT buffer containing 0.55 μg/mL iloprost, 0.28 mM RGDW and 800 μg/mL ristocetin. After that, another 220 μL of HT buffer containing 0.55 μg/mL iloprost, 0.28 mM RGDW and 800 μg/mL ristocetin was added and agglutinates were placed back into the aggregometer. Plasmin (50 μL, 420 μg/mL working stock, final concentration 75 μg/mL) was added and subsequently inactivated with PPACK (5 μL, 12.5 mM working stock, 217.8 UM final concentration) at indicated time points. Samples were spun down for 15 min at 400×g and supernatants were collected for further analysis by Western blot and ELISA.

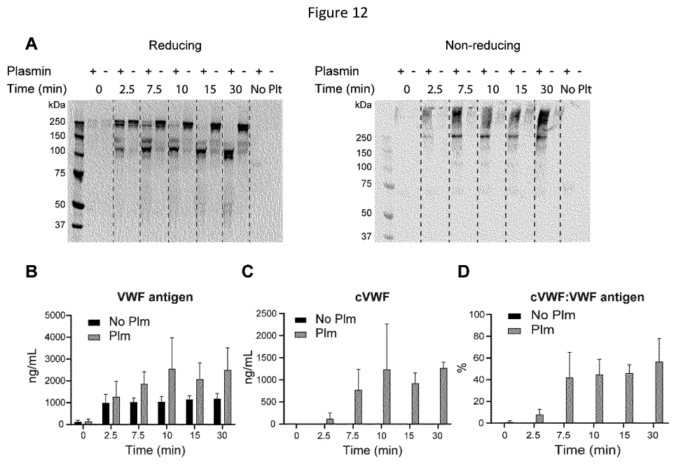

Control samples without plasmin show initial spontaneous VWF release from these complexes (approximately 1 μg/mL; FIG. 12A,B). In the presence of plasmin, this is 2 μg/mL after ˜10 minutes and this additional VWF is in a cleaved state (FIG. 12A-C). Approximately 50% of the VWF that is released in these experiments is cleaved (FIG. 12D). These findings indicate that soluble cVWF is released from microthrombi during degradation by plasmin.

### Example 5—Progressive cVWF Formation by Shear-Unfolded VWF Under Flow

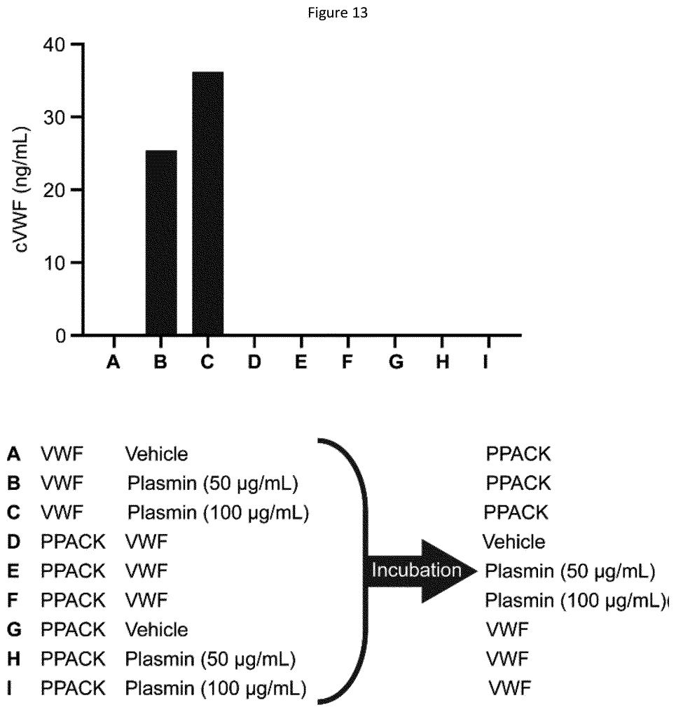

The previous experiments were performed under static conditions, using ristocetin to unfold VWF. We next sought to determine whether cVWF is also formed immediately after VWF secretion from endothelial cells under flow. We therefore perfused washed platelets over histamine-stimulated HUVECs to form stable platelet-covered VWF strings. These strings were allowed to stabilize for ten minutes and subsequently exposed to plasmin under flow. Samples were collected in PPACK to protect VWF from cleavage (FIG. 13). The time course of one experiment is 13.5 minutes. One sample was taken prior to perfusion of plasmin or medium control to assess (c) VWF baseline levels.

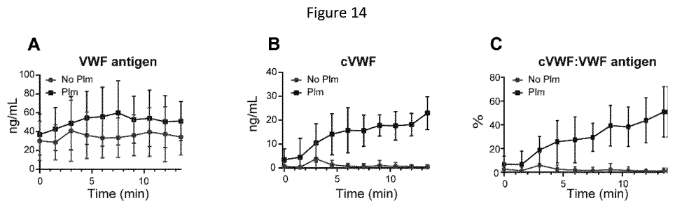

Total VWF release was stable throughout experiments and comparable between plasmin-treated and control samples (30-50 ng/ml; FIG. 14A). In the presence of plasmin, cVWF forms progressively, reaching a maximum concentration of ˜20 ng/ml (FIG. 14B). Similar to the static experiments in FIG. 7, ˜50% of VWF is cleaved in this single-pass flow model (FIG. 14C).

### Example 6—cVWF Formation During TTP Attacks in Human Patients

Our in vitro findings show that cVWF is released during microthrombus degradation by plasmin. We next set out to investigate whether this also takes place in TTP patients in vivo.

Citrated plasmas were obtained under informed consent from 10 healthy volunteers, 26 patients with acute TTP (collected before transfusion), and 20 TTP patients in clinical remission (collected between one month and six years after their most recent TTP attack) with ethical approval of the University Medical Center Utrecht. Citrated plasma samples from 20 patients in remission without clinical signs of microangiopathy were collected between one month and six years after their most recent TTP attack. Study cohort is described in Table 1.

TABLE 1
Patient cohort for study of cVWF formation during TTP attacks

| | N= | Gender (% Female) | Age (median (IQR)) |
|---|---|---|---|
| Acute Attack | 26 | 77 | 42 (37.5-52) |
| Remission TTP | 20 | 88 | 57 (39.5-64.5) |
| Healthy donors | 10 | 70 | 34 (28-45.5) |

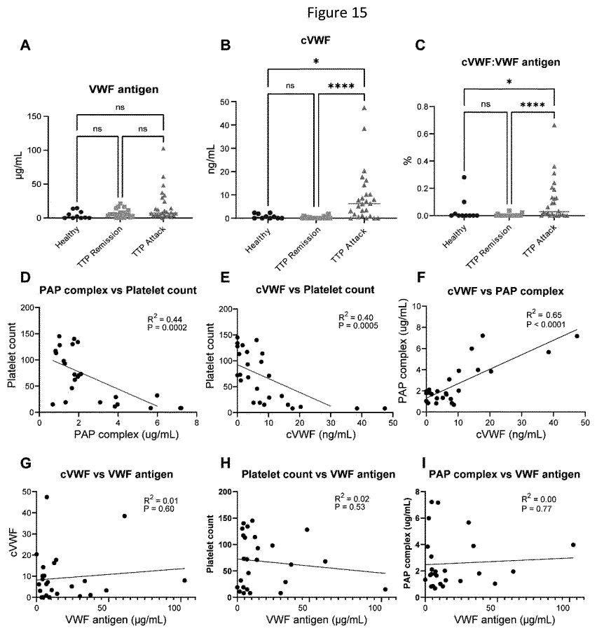

We first observed a trend where VWF antigen levels appear to increase in a subset of patients during TTP attacks (FIG. 15A). This suggests ongoing endothelial injury or lack of VWF metabolism, but fails to reach statistical significance. In contrast, cVWF levels are significantly increased in attack samples (FIG. 15B), but unlike in vitro studies, on average only 0.1% of the total plasma pool is in a plasmin-cleaved state (FIG. 15C).

Plasmin-α2-antiplasmin (PAP) complexes were determined by ELISA according to the manufacturer's protocol (Technoclone, Vienna, Austria). Platelet counts were determined by hemocytometry (CELLDYN Emerald Hematology Analyzer, Abbott Laboratories, Chicago, Illinois, USA). Plasma samples were diluted eight times in block buffer for evaluation of cVWF levels by ELISA. Samples were diluted 640 times in block buffer for assessment of total VWF antigen. Assay standards were spiked with citrated normal pooled plasma (1:8 for cVWF and 1:640 for intact VWF). Results were analysed by Kruskall-Wallis test followed by Dunn's multiple comparisons test. Correlations were computed by Pearson's correlation coefficients.

It was previously reported that plasmin-α2-antiplasmin (PAP) complex levels, a marker for plasminogen activation, are increased in TTP patients during attacks. In our study cohort, there is moderate negative correlation between PAP complexes and platelet counts. Patients with acute attacks generally have deep thrombocytopenia and elevated PAP-complex levels in plasma (FIG. 15D; R2=0.44). Accordingly, the thrombocytopeniatients have increased cVWF levels (FIG. 15E; R2=0.40). The presence of increased PAP-levels strongly correlates with cVWF levels (FIG. 15F), indicating that plasmin is likely responsible for VWF cleavage in vivo during TTP attacks. There is no correlation between VWF antigen levels and cVWF levels, platelet counts or PAP complex levels (FIG. 15G-I, respectively). This implies that thrombotic microangiopathy, rather than increased VWF levels, are the driving force behind endogenous cVWF production.

### Example 7—Therapeutic Plasminogen Activation Accelerates cVWF Production in a TTP Mouse Model

We next explored the impact of therapeutic plasminogen activation on cVWF production in a mouse model for TTP.

Animal studies were performed at the Animal Research Center of KU Leuven in accordance with protocols approved by the Institutional Animal Care and Use Committee. Female and male Adamts13−/− mice (CASA/Rk-C57BL/6J-129X1/SvJ background) of eight to twelve weeks old were used. Blood was collected at baseline (seven days prior to TTP challenge) in trisodium citrate (5:1 v/v of blood: 3.2% w/v trisodium citrate). To induce TTP, mice were anesthetized using isoflurane and intravenously injected with 2250 U/kg recombinant human VWF (Takeda, Vienna, Austria). After 15 min, mice were given an intravenous injection with saline or 40 μg/mouse of Microlyse. Citrated plasma samples were obtained at one, two, four or 24 hours after injection. Samples were diluted eight times in block buffer for assessment in the cVWF capture ELISA. For evaluation of total VWF levels, samples were diluted 16384 times in block buffer and subjected to VWF antigen ELISA. cVWF and intact VWF, respectively, were used as reference standards. Assay standards were diluted in block buffer spiked with pooled citrated plasma from healthy control C57BL/6 mice, in the same ratio as the samples (1:8 for cVWF reference standard and 1:16384 for VWF antigen reference standard). Results were analysed by two-way ANOVA followed by Šidák's post-hoc test.

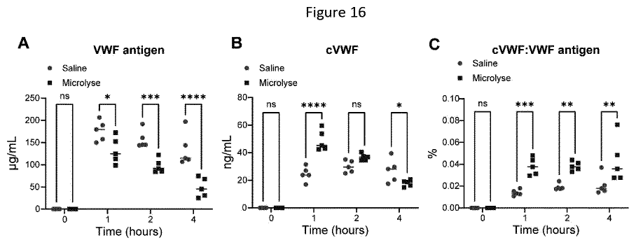

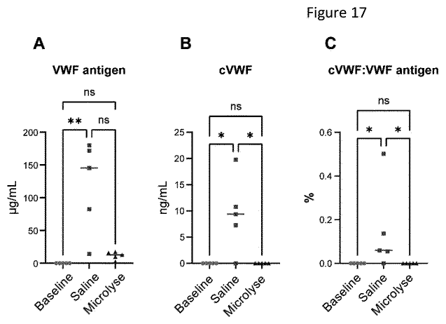

We found that Microlyse treatment leads to progressive VWF clearance compared to saline administration (FIG. 16A). This is accompanied by a steep rise in cVWF formation after one hour (FIG. 16B). Similar to human TTP patients (FIG. 15), endogenous cVWF production takes place in saline-treated mice. During these experiments, the fraction of VWF in a plasmin-cleaved state is approximately twice as high in Microlyse-treated mice compared to saline controls (FIG. 16C). After 24 hours, the administered rhVWF has been fully cleared in Microlyse-treated mice and cVWF is no longer present (FIG. 17). In contrast, both are present in saline-treated mice. Together, these data indicate that therapeutic plasminogen activation accelerates rhVWF clearance, which is accompanied by increased cVWF formation.

### Discussion

In the present study, we describe the identification of plasmin-cleaved VWF as a biomarker for degradation of microthrombi. We generated a VHH-based bioassay and characterized it in vitro, in biochemical and cell biological experiments. With this assay, we subsequently demonstrated that plasmin-cleaved VWF formation takes place in TTP patients during attacks of thrombotic microangiopathy. Furthermore, we show that therapeutic plasminogen activation is accompanied by increased cVWF production.

Circulating VWF is predominantly secreted by endothelial cells. The metalloprotease ADAMTS13 regulates its thrombogenicity by cleavage of its A2 domain. We show that the assay displays a strong preference for cVWF over ADAMTS13-cleaved VWF. This confirms that the cleavage product resulting from plasmin-mediated VWF degradation is biochemically distinct from ADAMTS13-cleaved VWF.

We observed that cVWF is not detectable in normal plasma or in the plasma of TTP patients during remission. This strengthens the notion that cVWF is only formed during conditions of severe endothelial distress. Accordingly, cVWF levels are elevated in TTP patients during acute attacks, and correlate with disease activity. In these patients, approximately 0.1% of all circulating VWF exists in a plasmin-cleaved state. This is much lower than the cVWF levels observed in our agglutination and single-pass flow studies, which reach up to 50% of the total VWF concentration. A possible explanation may be that these in vitro studies involve relatively high plasmin concentrations in the absence of physiological natural inhibitors that control plasminogen activation. Furthermore, there are no clearance mechanisms present. In addition, it is likely that plasminogen activation during endothelial distress occurs locally, proportionately resulting in low circulating levels of cVWF. In good correspondence, VWF antigen levels remain high in acute TTP patients with deep thrombocytopenia, suggesting that only a small fraction of circulating VWF actively contributes to microvascular occlusions. A long circulating half-life is an advantageous property for biomarkers (PMID: 29214154) This for example is the case for D-dimer, but this biomarker lacks specificity towards a pathological state. Our studies in Adamts13−/− mice confirm that therapeutic plasminogen activation with Microlyse leads to rapid clearance of the administered recombinant human VWF (rhVWF). Only a small fraction of the remaining rhVWF circulates in a cleaved state during this process, suggesting that cVWF is relatively short-lived. Our observations in TTP patients show comparable endogenous cVWF levels during attacks, despite the fact that their VWF antigen levels are not raised to the extent that they are in the TTP mouse model. These combined findings suggest that, in contrast to D-dimer, cVWF has positive discriminatory properties that are highly specific for an ongoing localized disease process.

VWF is regarded as a driving factor in a multitude of pathologies that involve endothelial dysfunction (PMID: 16706957). It is therefore conceivable that cVWF formation also occurs in other forms of thrombotic microangiopathy or even fibrin-dependent forms of thrombosis with an imbalance between VWF and ADAMTS13. This skewed balance is known to occur in transplant associated thrombotic microangiopathy (PMID: 32088973) in septic and non-septic intensive care unit (ICU) patients (PMID: 33606732) and during SARS-CoV-2 infection (PMID: 33230904). A lack of sufficient VWF modulation is also a known important contributor to more commonly occurring thrombotic events of the macrovasculature, such as acute ischemic stroke and VTE (PMID: 20798373; PMID: 36736832).

Taken together, we propose that cVWF is a cleavage product of VWF that reflects microvascular occlusion.

TABLE 2
Sequences of CDR regions of exemplary single
domain antibodies of the present invention

| VHH | CDR1 | | CDR2 | | CDR3 | |
|---|---|---|---|---|---|---|
| ID | SEQ ID NO. | | SEQ ID NO. | | SEQ ID NO. | |
| VHH#1 | TYTGG | 1 | TFSWNSGRT | 2 | HRTSLT | 3 |
| (F5) | | | HFADSVKG | | DYSFRY | |
| VHH#2 | NSMA | 4 | AISWNSGRT | 5 | HRVRIE | 6 |
| (G5) | | | DYADSVKG | | PKEYLY | |

TABLE 3
Sequences of exemplary single domain
antibodies of the present invention.

| | | SEQ |
|---|---|---|
| Clone | | ID |
| ID | Sequence | NO. |
| VHH#1 | EVQLVESGGGLVQAGGSLRLSCAASGQTDSTY | 7 |
| (F5) | TGGWFRQAPGKEREFVATESWNSGRTHFADSV | |
| | KGRFTISRDNAKSTVFLQMNSLKPEDTAVYYC | |
| | AAHRTSLTDYSFRYWGQGTHVTVSS | |
| VHH#2 | EVQLVESGGGLVQAGGSLRLSCAVSGRTVSNS | 8 |
| (G5) | MAWFRQAPEKEREFVAAISWNSGRTDYADSVK | |
| | GRFTISRDNAKNTVDLQMNSLKAEDTAVYYCA | |
| | VHRVRIEPKEYLYWGQGTQVTVSS | |
| Negative | QVQLQESGGGLVQAGGSLRLSCAASGRATSGH | 9 |
| control | GHYGMGWFRQVPGKEREFVAAIRWSGKETWYK | |
| VHH | DSVKGRFTISRDNAKTTVYLQMNSLKPEDTAV | |
| | YYCAARPVRVDDISLPVGFDYWGQGTQVTVSS | |

### Additional Paragraphs

According to certain aspects of the present invention there are provided single variable domains, single domain antibodies, polypeptides, nucleic acids, kits, methods and uses in accordance with the following paragraphs:

1 A single variable domain comprising a combination of complementary determining region (CDR) sequences, wherein the CDR sequences comprises a CDR3 sequence having at least 95% sequence identity with the amino acid sequence of SEQ ID NO: 6 or the amino acid sequence of SEQ ID NO:3.

2 The single variable domain according to paragraph 1, wherein the CDR sequences further comprises a CDR1 sequence having at least 95% sequence identity with the amino acid sequence of SEQ ID NO: 4 or the amino acid sequence of SEQ ID NO:1.

3 The single variable domain according to paragraph 1 or 2, wherein the CDR sequences further comprises a CDR2 sequence having at least 95% sequence identity with the amino acid sequence of SEQ ID NO: 5 or the amino acid sequence of SEQ ID NO:2.

4 The single variable domain according to paragraph 1, wherein the CDR sequences comprises:
a) a CDR3 sequence having at least 95% sequence identity with the amino acid sequence of SEQ ID NO: 6; and
b) a CDR1 sequence having at least 95% sequence identity with the amino acid sequence of SEQ ID NO: 4; and/or a CDR2 sequence having at least 95% sequence identity with the amino acid sequence of SEQ ID NO: 5.

5 The single variable domain according to paragraph 1, wherein the CDR sequences comprises:
a) a CDR3 sequence having at least 95% sequence identity with the amino acid sequence of SEQ ID NO: 3; and
b) a CDR1 sequence having at least 95% sequence identity with the amino acid sequence of SEQ ID NO: 1; and/or a CDR2 sequence having at least 95% sequence identity with the amino acid sequence of SEQ ID NO: 2.

6 The single variable domain according to paragraph 1, comprising or consisting of an amino acid sequence that has at least 95% sequence identity with SEQ ID NO: 8, or an amino acid sequence that has at least 95% sequence identity with SEQ ID NO: 7.

7 The single variable domain according to any one of paragraphs 1 to 6, wherein the single variable domain specifically binds to plasmin cleaved von Willebrand factor (VWF),

8 The single variable domain according to paragraph 7, wherein the affinity of the single variable domain for plasmin cleaved VWF is at least 100-fold higher than that for uncleaved VWF.

9 The single variable domain according to paragraph 7 or 8, wherein the affinity of the single variable domain for plasmin cleaved VWF is at least 10-fold higher than that for ADAMTS13 cleaved VWF.

10 The single variable domain according to any one of paragraphs 7 to 9, wherein the VWF is a human VWF.

11 A single domain antibody comprising or consisting of the single variable domain according to any one of paragraphs 1 to 9, wherein the single domain antibody is derived from a non-human source, preferably a camelid, more preferably a camel or llama monoclonal single domain antibody.

12 The single variable domain according to any one of paragraphs 1 to 10, or the single domain antibody according to paragraph 11, wherein the single variable domain or the single domain antibody is humanised or deimmunized.

13 A polypeptide comprising the single variable domain according to any one of paragraphs 1 to 9.

14 The single variable domain according to any one of paragraphs 1 to 10 or paragraph 12, or the single domain antibody according to paragraph 11 or 12, or the polypeptide according to paragraph 13, for use as a biomarker for plasmin cleaved VWF, or for use in a bioassay detecting, quantitatively or qualitatively, plasmin cleaved VWF.

15 An in vitro method for detecting plasmin cleaved VWF in a subject, or for determining the activity of a therapeutic agent regulating plasmin-mediated cleavage of VWF in a subject, the method comprising:
i) providing a sample from the subject;
ii) contacting the sample with the single variable domain according to any one of paragraphs 1 to 10 or paragraph 12, or the single domain antibody according to paragraph 11 or 12, or the polypeptide according to paragraph 13; and
iii) analysing the product of step ii) to determine the presence and/or the amount of plasmin cleaved VWF contained in the sample.

16 A method for determining the occurrence of a vascular event in a subject, the method comprising:
i) assaying a sample from said subject with the single variable domain according to any one of paragraphs 1 to 10 or paragraph 12, or the single domain antibody according to paragraph 11 or 12, or the polypeptide according to paragraph 13, to determine a concentration of plasmin cleaved VWF contained in the sample; and
ii) determining the occurrence of a vascular event in the subject if the concentration of plasmin cleaved VWF is at least 4 ng/mL.

17 The method according to paragraph 15 or 16, wherein the sample is the sample is a blood sample, a serum sample, a blood plasma sample, or a urine sample.

18 The method according to any one of paragraphs 15 to 17, wherein the subject is a human, a mice, a rat, or a guinea pig.

19 The method according to any one of paragraphs 16 to 17, wherein the vascular event is a disease or condition selected from the group consisting of: acquired or hereditary thrombotic thrombocytopenia purpura (TTP) complement-mediated thrombotic microangiopathy, haemolytic uremic syndrome, antiphospholipid antibody syndrome, non-occlusive thrombosis, the formation of an occlusive thrombus, arterial thrombus formation, acute coronary occlusion, peripheral arterial occlusive disease, restenosis and disorders arising from coronary by-pass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, transient cerebral ischemic attack, unstable or stable angina pectoris, cerebral infarction, HELLP syndrome, carotid endarterectomy, carotid artery stenosis, critical limb ischemia, cardioembolism, peripheral vascular disease, restenosis, sickle cell disease myocardial infarct, a bleeding episode, acute ischemic stroke, pulmonary embolism, Chronic thromboembolic pulmonary hypertension, SEPSIS, COVID-19 and transplant associated thrombotic microangiopathy, medical device-associated thrombosis, acute- or chronic liver disease.

20. A nucleic acid encoding the polypeptide according to paragraph 13.

21. A Kit comprising:
a) an element comprising the single variable domain according to any one of paragraphs 1 to 10 or paragraph 12, or the single domain antibody according to paragraph 11 or 12, or the polypeptide according to paragraph 13; and
b) means for detecting the binding of the element to plasmin-cleaved VWF; and, optionally,
c) means for collecting a sample from a subject.

1. A single variable domain comprising a combination of complementary determining region (CDR) sequences, wherein the CDR sequences comprises a CDR3 sequence having at least 95% sequence identity with the amino acid sequence of SEQ ID NO: 6 or the amino acid sequence of SEQ ID NO:3.

2. The single variable domain according to claim 1, wherein the CDR sequences further comprises a CDR1 sequence having at least 95% sequence identity with the amino acid sequence of SEQ ID NO: 4 or the amino acid sequence of SEQ ID NO:1; and/or wherein the CDR sequences further comprises a CDR2 sequence having at least 95% sequence identity with the amino acid sequence of SEQ ID NO: 5 or the amino acid sequence of SEQ ID NO:2.

3. The single variable domain according to claim 1, wherein the CDR sequences comprises a combination of:
a) a CDR3 sequence having at least 95% sequence identity with the amino acid sequence of SEQ ID NO: 6; and
b) a CDR1 sequence having at least 95% sequence identity with the amino acid sequence of SEQ ID NO: 4; and/or a CDR2 sequence having at least 95% sequence identity with the amino acid sequence of SEQ ID NO: 5;
or a combination of:
a) a CDR3 sequence having at least 95% sequence identity with the amino acid sequence of SEQ ID NO: 3; and
b) a CDR1 sequence having at least 95% sequence identity with the amino acid sequence of SEQ ID NO: 1; and/or a CDR2 sequence having at least 95% sequence identity with the amino acid sequence of SEQ ID NO: 2.

4. The single variable domain according to claim 1, comprising or consisting of an amino acid sequence that has at least 95% sequence identity with SEQ ID NO: 8, or an amino acid sequence that has at least 95% sequence identity with SEQ ID NO: 7.

5. The single variable domain according to any one of claims 1 to 4, wherein the single variable domain specifically binds to plasmin cleaved von Willebrand factor (VWF), preferably a human VWF.

6. The single variable domain according to claim 5, wherein the affinity of the single variable domain for plasmin cleaved VWF is at least 100-fold higher than that for uncleaved VWF; and/or wherein the affinity of the single variable domain for plasmin cleaved VWF is at least 10-fold higher than that for ADAMTS13 cleaved VWF.

7. A single domain antibody comprising or consisting of the single variable domain according to any one of claims 1 to 6, wherein the single domain antibody is derived from a non-human source, preferably a camelid, more preferably a camel or llama monoclonal single domain antibody.

8. A polypeptide comprising the single variable domain according to any one of claims 1 to 6.

9. The single variable domain according to any one of claims 1 to 6, or the single domain antibody according to claim 7, or the polypeptide according to claim 8, for use as a biomarker for plasmin cleaved VWF, or for use in a bioassay detecting, quantitatively or qualitatively, plasmin cleaved VWF.

10. An in vitro method for detecting plasmin cleaved VWF in a subject, or for determining the activity of a therapeutic agent regulating plasmin-mediated cleavage of VWF in a subject, the method comprising:
i) providing a sample from the subject;
ii) contacting the sample with the single variable domain according to any one of claims 1 to 6, or the single domain antibody according to claim 7, or the polypeptide according to claim 8; and
iii) analysing the product of step ii) to determine the presence and/or the amount of plasmin cleaved VWF contained in the sample.

11. A method for determining the occurrence of a vascular event in a subject, the method comprising:
i) assaying a sample from said subject with the single variable domain according to any one of claims 1 to 6, or the single domain antibody according to claim 7, or the polypeptide according to claim 8, to determine a concentration of plasmin cleaved VWF contained in the sample; and
ii) determining the occurrence of a vascular event in the subject if the concentration of plasmin cleaved VWF is at least 4 ng/ml.

12. The method according to claim 10 or 11, wherein the sample is the sample is a blood sample, a serum sample, a blood plasma sample, or a urine sample; and/or wherein the subject is a human, a mice, a rat, or a guinea pig.

13. The method according to claim 11 or 12, wherein the vascular event is a disease or condition selected from the group consisting of: acquired or hereditary thrombotic thrombocytopeni purpura (TTP) complement-mediated thrombotic microangiopathy, haemolytic uremic syndrome, antiphospholipid antibody syndrome, non-occlusive thrombosis, the formation of an occlusive thrombus, arterial thrombus formation, acute coronary occlusion, peripheral arterial occlusive disease, restenosis and disorders arising from coronary by-pass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, transient cerebral ischemic attack, unstable or stable angina pectoris, cerebral infarction, HELLP syndrome, carotid endarterectomy, carotid artery stenosis, critical limb ischemia, cardioembolism, peripheral vascular disease, restenosis, sickle cell disease myocardial infarct, a bleeding episode, acute ischemic stroke, pulmonary embolism, Chronic thromboembolic pulmonary hypertension, SEPSIS, COVID-19 and transplant associated thrombotic microangiopathy, medical device-associated thrombosis, acute- or chronic liver disease.

14. A nucleic acid encoding the polypeptide according to claim 8.

15. A Kit comprising:
a) an element comprising the single variable domain according to any one of claims 1 to 6, or the single domain antibody according to claim 7, or the polypeptide according to claim 8; and
b) means for detecting the binding of the element to plasmin-cleaved VWF; and, optionally,
c) means for collecting a sample from a subject.
